SEQUENCE LISTING (15 sequences)

SEQ ID NO: 1 - DNA, 29 bp, synthetic construct
  source 1..29: mol_type = other DNA
  modified_base 23: mod_base = OTHER; note = uracil
aatgatacggcgaccaccgagatctacac

SEQ ID NO: 2 - DNA, 24 bp, synthetic construct
  modified_base 22: mod_base = OTHER; note = 8-oxoguanine
  source 1..24: mol_type = other DNA
caagcagaagacggcatacganat

SEQ ID NO: 3 - DNA, 24 bp, synthetic construct
  modified_base 20: mod_base = OTHER; note = 8-oxoguanine
  source 1..24: mol_type = other DNA
caagcagaagacggcatacnagat

SEQ ID NO: 4 - DNA, 24 bp, synthetic construct
  modified_base 20: mod_base = OTHER; note = 8-oxoguanine
  modified_base 22: mod_base = OTHER; note = 8-oxoguanine
  source 1..24: mol_type = other DNA
caagcagaagacggcatacnanat

SEQ ID NO: 5 - DNA, 29 bp, synthetic construct
  modified_base 23: mod_base = OTHER; note = allyl-T
  source 1..29: mol_type = other DNA
aatgatacggcgaccaccgaganctacac

SEQ ID NO: 6 - DNA, 35 bp, synthetic construct
  source 1..35: mol_type = other DNA
gctggcacgtccgaacgcttcgttaatccgttgag

SEQ ID NO: 7 - DNA, 35 bp, synthetic construct
  source 1..35: mol_type = other DNA
ctcaacggattaacgaagcgttcggacgtgccagc

SEQ ID NO: 8 - DNA, 35 bp, synthetic construct
  source 1..35: mol_type = other DNA
cgtcgtctgccatggcgcttcggtggatatgaact

SEQ ID NO: 9 - DNA, 35 bp, synthetic construct
  source 1..35: mol_type = other DNA
agttcatatccaccgaagcgccatggcagacgacg

SEQ ID NO: 10 - DNA, 35 bp, synthetic construct
  source 1..35: mol_type = other DNA
acggccgctaatatcaacgcgtcgaatccgcaact

SEQ ID NO: 11 - DNA, 35 bp, synthetic construct
  source 1..35: mol_type = other DNA
agttgcggattcgacgcgttgatattagcggccgt

SEQ ID NO: 12 - DNA, 34 bp, synthetic construct
  source 1..34: mol_type = other DNA
gccgcgttacgttagccggactattcgatgcagc

SEQ ID NO: 13 - DNA, 34 bp, synthetic construct
  source 1..34: mol_type = other DNA
gctgcatcgaatagtccggctaacgtaacgcggc

SEQ ID NO: 14 - DNA, 24 bp, synthetic construct
  source 1..24: mol_type = other DNA
aggaggaggaggaggaggaggagg

SEQ ID NO: 15 - DNA, 24 bp, synthetic construct
  source 1..24: mol_type = other DNA
cctcctcctcctcctcctcctcct

(19) United States
(12) Patent Application Publication
(10) Pub. No.: US 2023/0302422 A1
(43) Pub. Date: Sep. 28, 2023

(54) CHEMICAL PLANAR ARRAY

(71) Applicants: ILLUMINA, INC., San Diego, CA (US); Illumina Singapore Pte. Ltd., Singapore (SG)

(72) Inventors: Zhong Mei, San Diego, CA (US); Adithya Ramanitharapandian, Singapore (SG); Zhonghuan Luo, San Diego, CA (US); Hui Han, San Diego, CA (US)

(21) Appl. No.: 18/187,608

(22) Filed: Mar. 21, 2023

Related U.S. Application Data
(60) Provisional application No. 63/322,556, filed on Mar. 22, 2022.

(51) Int. Cl. B01J 19/00; C12Q 1/6874

(52) U.S. Cl. CPC B01J 19/0046; C12Q 1/6874; B01J 2219/00596; G03F 7/0002

(57) ABSTRACT

A sequencing kit includes a plurality of particles and a flow cell. The plurality of particles includes a primer set attached to a surface of each of the plurality of particles; and a flow cell surface attachment mechanism attached to the surface of each of the plurality of particles. The flow cell surface attachment mechanism is selected from the group consisting of a capture primer, an alkene, an alkyne, biotin, and a charged polymer. The flow cell includes a plurality of chemical pads that are spatially separated from one another on a substantially flat substrate surface, each of the chemical pads including chemistry to attach to the surface attachment mechanism.

### CROSS REFERENCE TO RELATED APPLICATION

This application claims the benefit of U.S. Provisional Application Ser. No. 63/322,556, filed Mar. 22, 2022, the contents of which is incorporated by reference herein in its entirety.

### REFERENCE TO SEQUENCE LISTING

The Sequence Listing submitted herewith is hereby incorporated by reference in its entirety. The name of the file is ILI226B_IP-2229-US_Sequence_Listing.xml, the size of the file is 17,549 bytes, and the date of creation of the file is Mar. 17, 2023.

### BACKGROUND

Various protocols in biological or chemical research involve performing a large number of controlled reactions on local support surfaces or within predefined reaction chambers. The designated reactions may then be observed or detected and subsequent analysis may help identify or reveal properties of chemicals involved in the reaction. In some examples, the controlled reactions alter charge, conductivity, or some other electrical property, and thus an electronic system may be used for detection. In other examples, the controlled reactions generate fluorescence, and thus an optical system may be used for detection.

### SUMMARY

Functionalized particles are disclosed herein. Each of the functionalized particles includes the chemistry for seeding and clustering library templates, and thus can be used as part of an off-flow cell workflow. Thus, the functionalized particles enable pre-clustered particles, including amplicons of the library templates, to be formed.

Flow cells for use with the pre-clustered particles are also disclosed herein. The flow cell substrate includes a planar array of chemical pads that are spatially separated from one another on the substantially flat surface of the substrate. These chemical pads can anchor the pre-clustered particles at predetermined locations along the substantially flat surface. The planar nature of the chemical pads enables a relatively high number of pre-clustered particles to be anchored, in part, because the pre-clustered particles do not have to fit into wells defined in the substrate. Moreover, well geometry can limit the sequencing reagent exchange and reaction rate. Because the functionalized particles are attached to the substantially flat surface rather than confined in wells, more of the particle surface, including the amplicons, is exposed to sequencing reagents. The high loading of the pre-clustered particles anchored to the flow cell substrate results in a high number of amplicons that are sequences, and the improved sequencing reagent accessibility results in amplified fluorescence signals during sequencing. Overall, the pre-clustered particles and the flow cell disclosed herein can help to improve sequencing metrics.

### INTRODUCTION

A first aspect disclosed herein is a sequencing kit, comprising: a plurality of particles including: a primer set attached to a surface of each of the plurality of particles, and a flow cell surface attachment mechanism attached to the surface of each of the plurality of particles, the flow cell surface attachment mechanism being selected from the group consisting of a capture primer, an alkene, an alkyne, biotin, and a charged polymer; and a flow cell including a plurality of chemical pads that are spatially separated from one another on a substantially flat substrate surface, each of the chemical pads including chemistry to attach to the surface attachment mechanism.

In an example of the first aspect, each of the plurality of particles includes a core and a hydrogel attached to the core; and the primer set is attached to the hydrogel.

In an example of the first aspect, the flow cell surface attachment mechanism of each of the plurality of particles is the alkene and alkyne; the alkene or the alkyne is a functional group of the hydrogel; and each of the plurality of chemical pads is a chemical capture agent.

In an example of the first aspect, the flow cell surface attachment mechanism of each of the plurality of particles is the capture primer; the capture primer is one of the primers of the primer set; each of the plurality of chemical pads is poly(N-(5-azidoacetamidylpentyl)acrylamide-co-acrylamide); and the sequencing kit further comprises an azide reducing agent to convert at least some azide groups of the poly(N-(5-azidoacetamidylpentyl)acrylamide-co-acrylamide) to amine groups that can attach to a 3′ end of the capture primer.

In an example of the first aspect, the flow cell surface attachment mechanism of each of the plurality of particles is the biotin; and each of the plurality of chemical pads is streptavidin.

In an example of the first aspect, the flow cell surface attachment mechanism of each of the plurality of particles is the charged polymer; and each of the plurality of chemical pads includes a counter ion of the charged polymer.

In an example of the first aspect, the charged polymer is selected from the group consisting of polylysine, polyethylenimine and polypeptide; and the counter ion is selected from the group consisting of oligonucleotide, polyacrylic acid and polystyrene sulfonate.

In an example of the first aspect, the flow cell surface attachment mechanism of each of the plurality of particles is the capture primer; the capture primer is AGGAGGAGGAGGAGGAGGAGGAGG; and each of the plurality of chemical pads includes a complementary primer of the capture primer.

It is to be understood that any features of the first aspect may be combined together in any desirable manner and/or may be combined with any of the examples disclosed herein to achieve the benefits as described in this disclosure, including, for example, enhanced fluorescence signals during imaging events in a sequencing protocol.

A second aspect disclosed herein is a method, comprising: amplifying a plurality of library fragments on respective surfaces of a plurality of particles, thereby generating pre-clustered particles, each of the plurality of particles including a surface attachment mechanism selected from the group consisting of a primer, an alkene, an alkyne, biotin, and a charged polymer; and introducing the pre-clustered particles to a flow cell including a plurality of chemical pads that are spatially separated from one another on a substantially flat substrate surface, each of the chemical pads including chemistry to attach to the surface attachment mechanism.

It is to be understood that any features of the second aspect may be combined together in any desirable manner. Moreover, it is to be understood that any combination of features of the first aspect and/or of the second aspect may be used together, and/or may be combined with any of the examples disclosed herein to achieve the benefits as described in this disclosure, including, for example, enhanced fluorescence signals during imaging events in a sequencing protocol.

A third aspect disclosed herein is a method, comprising: generating a plurality of chemical pads that are spatially separated from one another on a substantially flat surface of a substrate, wherein each of the chemical pads includes poly(N-(5-azidoacetamidylpentyl)acrylamide-co-acrylamide); and exposing the plurality of chemical pads to an azide reducing agent to convert at least some azide groups of the poly(N-(5-azidoacetamidylpentyl)acrylamide-co-acrylamide) to amine groups.

In an example of the third aspect, the azide reducing agent is selected from the group consisting of a phosphine and a phosphite.

In an example of the third aspect, the azide reducing agent is the phosphine selected from the group consisting of Tris(2-carboxyethyl)phosphine hydrochloride) (TCEP) and Tris(hydroxypropyl)phosphine; and exposing the plurality of chemical pads to the azide reducing agent takes place at a temperature ranging from about 50° C. to about 60° C. for a time ranging from about 5 minutes to about 10 minutes.

In an example of the third aspect, generating the plurality of chemical pads involves: applying a sacrificial layer over the substantially flat surface; applying a resin layer over the sacrificial layer; patterning the resin layer to include concave regions separated by convex regions; removing the resin layer and the sacrificial layer from the concave regions, thereby exposing the substantially flat surface at the concave regions; applying the poly(N-(5-azidoacetamidylpentyl)acrylamide-co-acrylamide) over the substantially flat surface at the concave regions and over the convex regions; and lifting off remaining portions of the sacrificial layer, thereby removing the resin layer and the poly(N-(5-azidoacetamidylpentyl)acrylamide-co-acrylamide) that overlie the sacrificial layer.

In an example of the third aspect, generating the plurality of chemical pads involves: applying the poly(N-(5-azidoacetamidylpentyl)acrylamide-co -acrylamide) over the substantially flat surface; applying a sacrificial layer over the poly(N-(5-azidoacetamidylpentyl)acrylamide-co-acrylamide); applying a resin layer over the sacrificial layer; patterning the resin layer to include concave regions separated by convex regions; removing the resin layer, the sacrificial layer, and the poly(N-(5-azidoacetamidylpentyl)acrylamide-co-acrylamide) from the concave regions, thereby exposing the substantially flat surface at the concave regions; and lifting off remaining portions of the sacrificial layer, thereby removing the resin layer that overlies the sacrificial layer.

In an example of the third aspect, removing the resin layer, the sacrificial layer, and the poly(N-(5-azidoacetamidylpentyl)acrylamide-co -acrylamide) from the concave regions involves: anisotropically etching the resin layer, the sacrificial layer, and the poly(N-(5-azidoacetamidylpentyl)acrylamide-co -acrylamide) from the concave regions; and generating an undercut profile by isotropically etching some of the sacrificial layer and the poly(N-(5-azidoacetamidylpentyl)acrylamide-co-acrylamide) that underlie the resin layer at the convex regions.

In an example of the third aspect, generating the plurality of chemical pads involves: applying the poly(N-(5-azidoacetamidylpentyl)acrylamide-co -acrylamide) over the substantially flat surface; applying a sacrificial layer over the poly(N-(5-azidoacetamidylpentyl)acrylamide-co-acrylamide); patterning the sacrificial layer to include concave regions separated by convex regions; removing the sacrificial layer and the poly(N-(5-azidoacetamidylpentyl)acrylamide-co -acrylamide) from the concave regions, thereby exposing the substantially flat surface at the concave regions; and lifting off remaining portions of the sacrificial layer.

In an example of the third aspect, generating the plurality of chemical pads involves: using photolithography to generate a plurality of sacrificial pads on the substantially flat surface such that regions of the substantially flat surface separate each of the plurality of sacrificial pads; applying the poly(N-(5-azidoacetamidylpentyl)acrylamide-co-acrylamide) on the plurality of spatially separated sacrificial pads and on the regions of the substantially flat surface; introducing ultraviolet light through the substrate, whereby portions of the poly(N-(5-azidoacetamidylpentyl)acrylamide-co-acrylamide) overlying the regions of the substantially flat surface are cured and other portions of the poly(N-(5-azidoacetamidylpentyl)acrylamide-co-acrylamide) overlying the plurality of spatially separated sacrificial pads are uncured; removing the uncured portions of the poly(N-(5-azidoacetamidylpentyl)acrylamide-co-acrylamide); and removing the plurality of sacrificial pads.

It is to be understood that any features of the third aspect may be combined together in any desirable manner. Moreover, it is to be understood that any combination of features of the first aspect and/or of the second aspect and/or of the third aspect may be used together, and/or may be combined with any of the examples disclosed herein to achieve the benefits as described in this disclosure, including, for example, enhanced fluorescence signals during imaging events in a sequencing protocol.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A is a schematic illustration of an example of a functionalized particle, i.e., the particle before it is exposed to clustering;

FIG. 1B is a schematic illustration of an example of a pre-clustered particle, i.e., a functionalized particle after it is exposed to clustering;

FIG. 2A is a top view of an example of a flow cell;

FIG. 2B is an enlarged, and partially cutaway view of one example of the flow cell architecture in a flow channel of the flow cell;

FIG. 3 is an enlarged, cross-sectional view, taken along the 2-2 line of FIG. 2A, depicting an example of the flow cell architecture including the pre-clustered particles anchored to flow cell chemical pads;

FIG. 4A through FIG. 4E are schematic views that together illustrate one example of a method to generate the flow cell architecture shown in FIG. 2B, where FIG. 4A depicts a sacrificial layer applied over a substrate, FIG. 4B depicts the patterning of a resin layer that is applied over the sacrificial layer of FIG. 4A, FIG. 4C depicts layers selectively etched from concave regions of the patterned resin layer of FIG. 4B to expose portions of the substrate, FIG. 4D depicts the deposition of the capture agent on the structure of FIG. 4C, and FIG. 4E depicts the removal of sacrificial layer and layers thereon;

FIG. 5A through FIG. 5E are schematic views that together illustrate another example of a method to generate the flow cell architecture shown in FIG. 2B, where FIG. 5A depicts a capture agent applied over a substrate, FIG. 5B depicts a sacrificial layer applied over the capture agent of FIG. 5A, FIG. 5C depicts the patterning of a resin layer that is applied over the sacrificial layer of FIG. 5B, FIG. 5D depicts layers selectively etched from concave regions of the patterned resin layer of FIG. 5C to expose portions of the substrate, and FIG. 5E depicts the removal of the sacrificial layer and layers thereon;

FIG. 5A through FIG. 5C, FIG. 5F, and FIG. 5G are schematic views that together illustrate yet another example of a method to generate the flow cell architecture shown in FIG. 2B, where FIG. 5A depicts a capture agent applied over a substrate, FIG. 5B depicts a sacrificial layer applied over the capture agent of FIG. 5A, FIG. 5C depicts the patterning of a resin layer that is applied over the sacrificial layer of FIG. 5B, FIG. 5F depicts layers selectively etched from concave regions of the patterned resin layer of FIG. 5C to generate undercut regions and expose portions of the substrate, and FIG. 5G depicts the removal of the sacrificial layer and layers thereon;

FIG. 6A through FIG. 6E are schematic views that illustrate still another example of a method to generate the flow cell architecture shown in FIG. 2B, where FIG. 6A depicts a capture agent applied over a substrate, FIG. 6B depicts a sacrificial layer applied over the capture agent of FIG. 6A, FIG. 6C depicts the patterning of the sacrificial layer of FIG. 6B, FIG. 6D depicts layers selectively etched from concave regions of the patterned sacrificial layer of FIG. 6D, and FIG. 6E depicts the removal of the sacrificial layer;

FIG. 7A through FIG. 7E are schematic views that together illustrate another example of a method to generate the flow cell architecture shown in FIG. 2B, where FIG. 7A depicts formation of sacrificial pads on a substrate, FIG. 7B depicts a photocurable capture agent applied over the sacrificial pads and the substrate of FIG. 7A, FIG. 7C depicts backside exposure to cure portions of the capture agent that do not overly the sacrificial pads, FIG. 7D depicts the removal of the uncured portions of the capture agent, and FIG. 7E depicts the removal of the sacrificial pads;

FIG. 8 is a scanning electron micrograph (SEM) showing a cross-sectional and perspective view to illustrate one example of a multi-layer stack of materials including a resin layer defining concave regions separated by convex regions;

FIG. 9A through FIG. 9C are a series of scanning electron micrographs showing a perspective view of one example of the multi-layer stack of FIG. 8 and after an etching time titration was performed to remove: interstitials of a resin layer (FIG. 9A, 9 minute etch); a sacrificial layer underlying the interstitials of the resin layer (FIG. 9B, 12 minute etch); and a functional layer underlying the sacrificial layer (Fig. C, 14 minute etch);

FIG. 10A through FIG. 10E are a series of scanning electron micrographs showing perspective views (FIG. 10A, 10B and 10D) and top views (FIG. 10C and FIG. 10E) of two different examples of a method for controlling the size of the chemical pads on a flow cell surface by tuning etching direction and time;

FIG. 11A through FIG. 11D are a series of scanning electron micrographs showing top views of one example of the flow cell architecture shown in FIG. 2B including the pre-clustered particles anchored to flow cell chemical pads; and

FIG. 11E through FIG. 11H are a series of scanning electron micrographs showing perspective views of one example of the flow cell architecture shown in FIG. 2B including the pre-clustered particles anchored to flow cell chemical pads.

### DETAILED DESCRIPTION

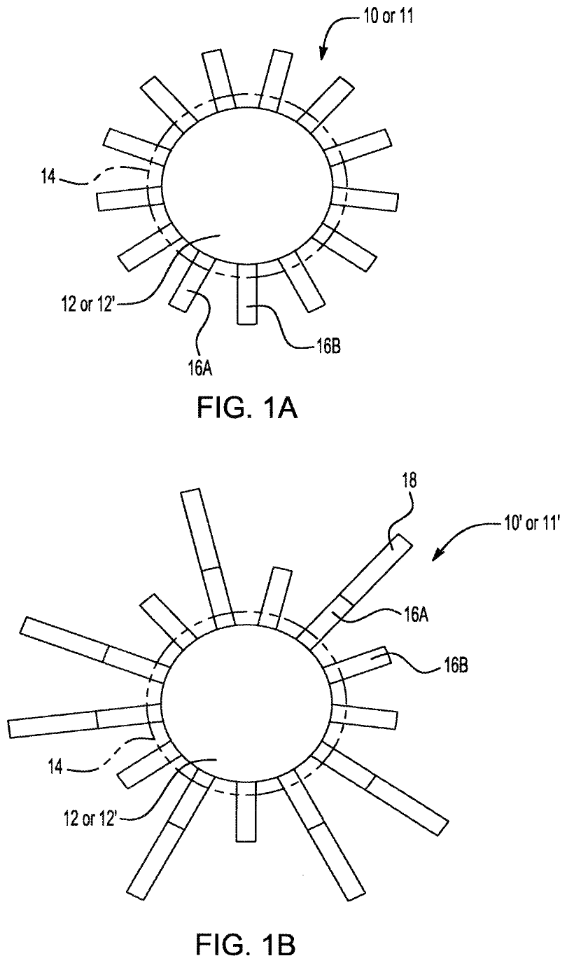

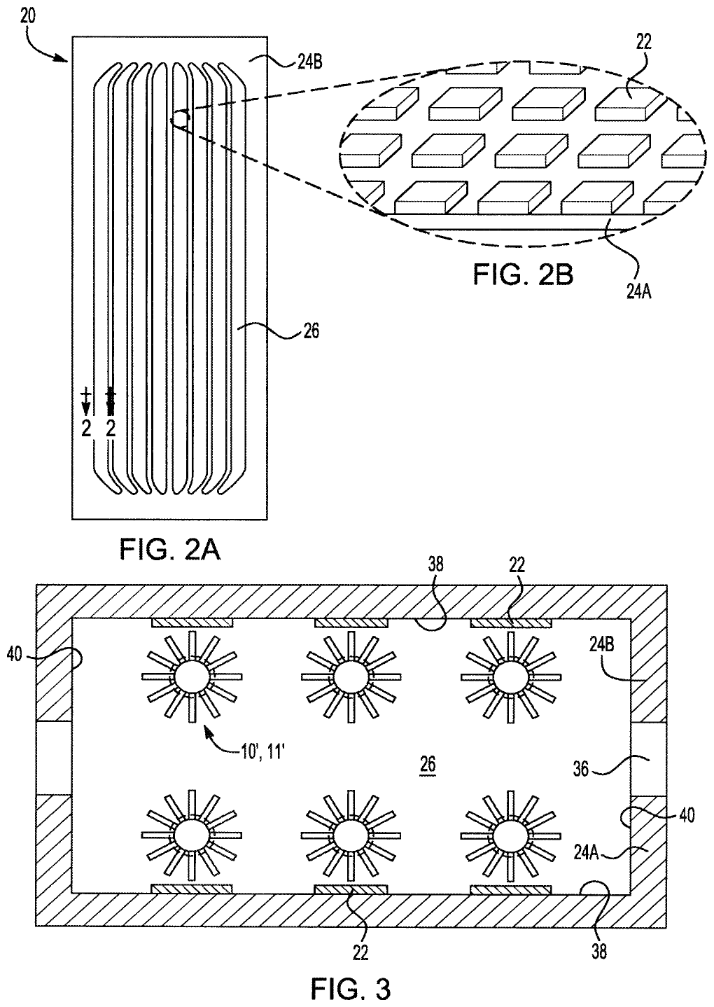

Sequencing kits comprising a plurality of particles and a flow cell are disclosed herein. Each of the particles includes the surface chemistry for seeding and clustering library templates as part of an off-flow cell workflow. The kit comprises (i) a plurality of particles 10 or 11 (shown in FIG. 1A), and (ii) a flow cell 20 (shown in FIG. 2A). In one example, the kit comprises (i) the plurality of particles 10, wherein each particle 10 includes a core 12, a hydrogel 14 attached to the core 12, a plurality of primers 16A, 16B attached to the hydrogel 14, and a flow cell surface attachment mechanism attached to the surface of the particle 10, and (ii) the flow cell 20, which includes a plurality of chemical pads 22, wherein each of the chemical pads 22 includes chemistry to attach to the surface attachment mechanism of the particles 10. In another example, the kit comprises (i) the plurality of particles 11, wherein each particle 11 includes a hydrogel core 12′, the plurality of primers 16A, 16B attached to the hydrogel core 12′, and a flow cell surface attachment mechanism attached to the surface of the particle 11, and (ii) the flow cell 20 including a plurality of chemical pads 22, wherein each of the chemical pads 22 includes chemistry to attach to the surface attachment mechanism of the particles 11. It is to be understood that the particle 11 does not include the hydrogel 14 at the surface of the hydrogel core 12′.

As mentioned, flow cells 20 for use with the particles 10, 11 (e.g., the pre-clustered particles 10′, 11′) are also disclosed herein. The flow cell 20 includes a substrate 24A that has a substantially flat surface. The substantially flat substrate surface of the flow cell 20 includes a plurality of chemical pads 22 that are spatially separated from one another along the substrate surface and that can anchor the pre-clustered particles 10′ or 11′ at predetermined locations along the substrate. Because the surface chemistry for seeding and clustering (e.g., primers 16A, 16B) is part of the particles 10 or 11 (used to generate the pre-clustered particles 10′, 11′), the flow cell substrate 24A is not exposed to primer grafting processes. The methods disclosed herein also do not involve polishing processes to create interstitial regions between the chemical pads 22. As such, the use of the particles 10, 10′ or 11, 11′ simplifies the flow cell substrate preparation process.

The chemical pads 22 enable a high number of pre-clustered particles 10′ or 11′ to be attached to the flow cell surface, and the attachment to the substantially flat surface enables greater exposure of sequencing reagents to the amplicons formed at the surface of the particles 10′ or 11′. Thus, during imaging events of a sequencing protocol, the pre-clustered particles 10′ or 11′ anchored to the flow cell substrate 24A can enhance optical signals.

### Definitions

It is to be understood that terms used herein will take on their ordinary meaning in the relevant art unless specified otherwise. Several terms used herein and their meanings are set forth below.

The singular forms “a”, “an”, and “the” include plural referents unless the context clearly dictates otherwise.

The terms comprising, including, containing and various forms of these terms are synonymous with each other and are meant to be equally broad.

The terms top, bottom, lower, upper, adjacent, on, etc. are used herein to describe the flow cell and/or the various components of the flow cell. It is to be understood that these directional terms are not meant to imply a specific orientation, but are used to designate relative orientation between components. The use of directional terms should not be interpreted to limit the examples disclosed herein to any specific orientation(s).

The terms first, second, etc. also are not meant to imply a specific orientation or order, but rather are used to distinguish one component from another.

An “acrylamide monomer” is a monomer with the structure

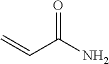

or a monomer including an acrylamide group. Examples of the monomer including an acrylamide group include azido acetamido pentyl acrylamide:

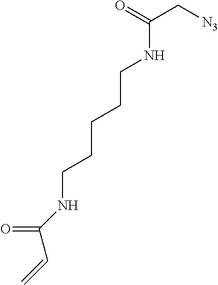 [SMILES: C=CC(=O)NCCCCCNC(=O)CN=[N+]=[N-]]

### and N-isopropylacrylamide:

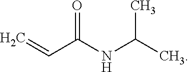

Other acrylamide monomers may be used.

An “aldehyde,” as used herein, is an organic compound containing a functional group with the structure —CHO, which includes a carbonyl center (i.e., a carbon double-bonded to oxygen) with the carbon atom also bonded to hydrogen and an R group, such as an alkyl or other side chain. The general structure of an aldehyde is:

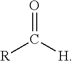

As used herein, “alkyl” refers to a straight or branched hydrocarbon chain that is fully saturated (i.e., contains no double or triple bonds). The alkyl group may have 1 to 20 carbon atoms. Example alkyl groups include methyl, ethyl, propyl, isopropyl, butyl, isobutyl, tertiary butyl, pentyl, hexyl, and the like. As an example, the designation “C1-4 alkyl” indicates that there are one to four carbon atoms in the alkyl chain, i.e., the alkyl chain is selected from the group consisting of methyl, ethyl, propyl, iso-propyl, n-butyl, isobutyl, sec-butyl, and t-butyl.

As used herein, “alkenyl” refers to a straight or branched hydrocarbon chain containing one or more double bonds. The alkenyl group may have 2 to 20 carbon atoms. Example alkenyl groups include ethenyl, propenyl, butenyl, pentenyl, hexenyl, and the like.

As used herein, “alkyne” or “alkynyl” refers to a straight or branched hydrocarbon chain containing one or more triple bonds. The alkynyl group may have 2 to 20 carbon atoms.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms. Examples of aryl groups include phenyl, naphthyl, azulenyl, and anthracenyl.

An “amino” functional group refers to an —NRₐR_{b} group, where Rₐ and R_{b} are each independently selected from hydrogen

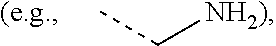

C1-6 alkyl, C2-6 alkenyl, C2-6 alkynyl, C3-7 carbocycle, C6-10 aryl, 5-10 membered heteroaryl, and 5-10 membered heterocyclyl, as defined herein.

As used herein, the terms “anchored” and “attached” refer to the state of two things being joined, fastened, adhered, connected or bound to each other, either directly or indirectly. For example, a primer can be attached to a hydrogel by a covalent or non-covalent bond. A covalent bond is characterized by the sharing of pairs of electrons between atoms. A non-covalent bond is a physical bond that does not involve the sharing of pairs of electrons and can include, for example, hydrogen bonds, ionic bonds, van der Waals forces, hydrophilic interactions and hydrophobic interactions. Other examples of attachment include magnetic attachment or electrostatic attachment.

An “azide” or “azido” functional group refers to —N₃.

As used herein, “carbocycle” means a non-aromatic cyclic ring or ring system containing only carbon atoms in the ring system backbone. When the carbocycle is a ring system, two or more rings may be joined together in a fused, bridged or spiro-connected fashion. Carbocycles may have any degree of saturation, provided that at least one ring in a ring system is not aromatic. Thus, carbocycles include cycloalkyls, cycloalkenyls, and cycloalkynyls. The carbocycle group may have 3 to 20 carbon atoms. Examples of carbocycle rings include cyclopropyl, cyclobutyl, cyclopentyl, cyclohexyl, cyclohexenyl, 2,3-dihydro-indene, bicyclo[2.2.2]octanyl, adamantyl, and spiro[ 4.4]nonanyl.

As used herein, the term “carboxylic acid” or “carboxyl” as used herein refers to —COOH.

A “chemical capture agent” is a material, molecule or moiety that is capable of anchoring to a surface attachment mechanism of a pre-clustered particle via a chemical mechanism, and may be a function of a chemical pad. One example chemical capture agent includes a primer (e.g., a PX primer) that is complementary to at least a portion of a capture primer attached to a pre-clustered particle. In some examples, the primer may be pre-grafted to a polymer (e.g., PAZAM) that can be deposited using an example of the method disclosed herein. Still another example chemical capture agent includes a member of a binding pair that is capable of binding to a second member of a binding pair that is attached to the pre-clustered particle. An example binding pair includes a streptavidin and biotin. Yet another example of the chemical capture agent is a chemical reagent capable of forming an electrostatic interaction or a covalent bond with the pre-clustered particles. Covalent bonds may be formed, for example, through click chemistry, amine-aldehyde coupling, amine-acid chloride reactions, etc.

A “chemical pad”, as used herein, refers to portion of a flow cell substrate having been modified chemically (e.g., to include a chemical capture agent) to allow for anchoring of a pre-clustered particle. In an example, the chemical pads may include chemistry to attach to the surface attachment mechanism that specifically binds, attaches, or is otherwise attracted (e.g., electrostatically, etc.) to the chemical pads on the flow cell surface.

As used herein, “cycloalkyl” refers to a completely saturated (no double or triple bonds) mono- or multi- cyclic hydrocarbon ring system. When composed of two or more rings, the rings may be joined together in a fused fashion. Cycloalkyl groups can contain 3 to 10 atoms in the ring(s). In some examples, cycloalkyl groups can contain 3 to 8 atoms in the ring(s). A cycloalkyl group may be unsubstituted or substituted. Example cycloalkyl groups include cyclopropyl, cyclobutyl, cyclopentyl, cyclohexyl, cycloheptyl, and cyclooctyl.

As used herein, “cycloalkenyl” or “cycloalkene” means a carbocycle ring or ring system having at least one double bond, wherein no ring in the ring system is aromatic. Examples include cyclohexenyl or cyclohexene and norbornenyl or norbornene. Also as used herein, “heterocycloalkenyl” or “heterocycloalkene” means a carbocycle ring or ring system with at least one heteroatom in ring backbone, having at least one double bond, wherein no ring in the ring system is aromatic.

As used herein, “cycloalkynyl” or “cycloalkyne” means a carbocycle ring or ring system having at least one triple bond, wherein no ring in the ring system is aromatic. An example is cyclooctyne. Another example is bicyclononyne. Also as used herein, “heterocycloalkynyl” or “heterocycloalkyne” means a carbocycle ring or ring system with at least one heteroatom in ring backbone, having at least one triple bond, wherein no ring in the ring system is aromatic.

The term “depositing,” as used herein, refers to any suitable application technique, which may be manual or automated, and, in some instances, results in modification of the surface properties. Generally, depositing may be performed using vapor deposition techniques, coating techniques, grafting techniques, or the like. Some specific examples include chemical vapor deposition (CVD), spray coating (e.g., ultrasonic spray coating), spin coating, dunk or dip coating, doctor blade coating, puddle dispensing, flow through coating, aerosol printing, screen printing, microcontact printing, inkjet printing, or the like.

The term “each,” when used in reference to a collection of items, is intended to identify an individual item in the collection, but does not necessarily refer to every item in the collection. Exceptions can occur if explicit disclosure or context clearly dictates otherwise.

The term “epoxy” (also referred to as a glycidyl or oxirane group) as used herein refers to

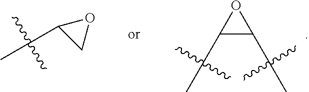

As used herein, the term “electrostatic capture agent” refers to a charged material that is a counter ion to a charged polymer of some examples of the pre-clustered particles disclosed herein. An example of an electrostatic capture agent is an electrode that can attract, when a proper voltage is applied, the charged polymer of the pre-clustered particles.

As used herein, the term “flow cell” is intended to mean a vessel having a flow channel where a reaction can be carried out, an inlet for delivering reagent(s) to the flow channel, and an outlet for removing reagent(s) from the flow channel. In some examples, the flow cell accommodates the detection of the reaction that occurs in the flow cell. For example, the flow cell can include one or more transparent surfaces allowing for the optical detection of arrays, optically labeled molecules, or the like.

As used herein, the term “flow cell chemical pad” or “chemical pad” refers to a discrete convex feature defined on a substrate surface and having a top surface to receive a pre-clustered particle and a base portion that is at least partially surrounded by interstitial region(s) of the substrate. As such, the convex features is a post, which can have any of a variety of shapes at the top portion including, as examples, round, elliptical, square, polygonal, star shaped (with any number of vertices), etc. The cross-section of a post taken orthogonally with the surface can be curved, square, polygonal, hyperbolic, conical, angular, etc.

As used herein, the term “flow cell surface attachment mechanism” or “surface attachment mechanism” or “mechanism” refers to a functional agent or a charged polymer that makes up or is attached to the core and/or the hydrogel or the hydrogel core in order to render the pre-clustered particle capable of anchoring to a chemical pad in a flow cell. The mechanism can be a material of the core and/or may be a functional agent that is part of or introduced to the hydrogel or the hydrogel core. The flow cell surface attachment mechanism can specifically bind, attach, or is otherwise be attracted (e.g., electrostatically, etc.) to the chemical pads on the flow cell surface.

As used herein, a “flow channel” or “channel” may be an area defined between two bonded components, which can selectively receive a liquid sample, reagents, etc. In some examples, the flow channel may be defined between two substrates, and thus may be in fluid communication with the pre-clustered particles anchored to each of the substrates. In other examples, the flow channel may be defined between a substrate and a lid, and thus may be in fluid communication with the pre-clustered particles anchored to the one substrate.

A “functional agent” is a material, molecule or moiety that is capable of anchoring to a chemical pad of a flow cell. One example functional agent includes a capture primer that is complementary to a primer (e.g., Px primer) on the flow cell chemical pad or that can attach to an amine functional group of the flow cell chemical pad. Another example functional agent includes a member of a binding pair that is capable of binding to a second member of a binding pair that is attached to the flow cell (e.g., biotin, streptavidin). Still another example of a functional agent is a functional group (e.g., an alkene, an alkyne) that is capable of covalently bonding to the flow cell chemical pad.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members.

As used herein, “heterocycle” means a non-aromatic cyclic ring or ring system containing at least one heteroatom in the ring backbone. Heterocycles may be joined together in a fused, bridged or spiro-connected fashion. Heterocycles may have any degree of saturation provided that at least one ring in the ring system is not aromatic. In the ring system, the heteroatom(s) may be present in either a non-aromatic or aromatic ring. The heterocycle group may have 3 to 20 ring members (i.e., the number of atoms making up the ring backbone, including carbon atoms and heteroatoms). In some examples, the heteroatom(s) are O, N, or S.

The term “hydrazine” or “hydrazinyl” as used herein refers to a —NHNH₂ group.

As used herein, the term “hydrazone” or “hydrazonyl” as used herein refers to a

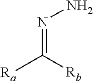

group in which Rₐ and R_{b} are each independently selected from hydrogen, C1-6 alkyl, C2-6 alkenyl, C2-6 alkynyl, C3-7 carbocycle, C6-10 aryl, 5-10 membered heteroaryl, and 5-10 membered heterocyclyl, as defined herein.

As used herein, “hydroxy” or “hydroxyl” refers to an —OH group.

The term “hydrogel” or “polymeric hydrogel” refers to a semi-rigid polymer that is permeable to liquids and gases. The hydrogel can swell when liquid (e.g., water) is taken up and that can contract when liquid is removed, e.g., by drying. While a hydrogel may absorb water, it is not water-soluble.

As used herein, the term “interstitial region” refers to an area, e.g., of a substrate that separates flow cell chemical pads or surrounds a lane. As an example, an interstitial region can separate one flow cell chemical pad of an array from another flow cell chemical pad of the array. As another example, an interstitial region can separate one lane of a flow cell from another lane of a flow cell. The flow cell chemical pads and lanes that are separated from each other can be discrete, i.e., lacking physical contact with each other. In many examples, the interstitial region is continuous, whereas the flow cell chemical pad or lanes are discrete, for example, as is the case for a plurality of lanes defined in or on an otherwise continuous surface. The separation provided by an interstitial region can be partial or full separation. Interstitial regions may have a surface material that differs from the surface material of the flow cell chemical pads or lanes. For example, flow cell chemical pads and lanes can have the hydrogel and primers therein or thereon, and the interstitial regions can be free of both the hydrogel and primers.

As used herein, a “negative photoresist” refers to a light sensitive material in which a portion that is exposed to light of particular wavelength(s) becomes insoluble to a developer. In these examples, the insoluble negative photoresist has less than 5% solubility in the developer. With the negative photoresist, the light exposure changes the chemical structure so that the exposed portions of the material becomes less soluble (than non-exposed portions) in the developer. While not soluble in the developer, the insoluble negative photoresist may be at least 99% soluble in a remover that is different from the developer. The remover may be a solvent or solvent mixture used, e.g., in a lift-off process.

In contrast to the insoluble negative photoresist, any portion of the negative photoresist that is not exposed to light is at least 95% soluble in the developer. This portion may be referred to as a “soluble negative photoresist”. In some examples, the soluble negative photoresist is at least 98%, e.g., 99%, 99.5%, 100%, soluble in the developer.

“Nitrile oxide,” as used herein, means a “RₐC≡N⁺O⁻” group in which Rₐ is defined herein. Examples of preparing nitrile oxide include in situ generation from aldoximes by treatment with chloramide-T or through action of base on imidoyl chlorides [RC(Cl)═NOH] or from the reaction between hydroxylamine and an aldehyde.

“Nitrone,” as used herein, means a

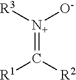

group in which R¹, R², and R³ may be any of the Rₐ and R_{b} groups defined herein, except that R³ is not hydrogen (H).

As used herein, a “nucleotide” includes a nitrogen containing heterocyclic base, a sugar, and one or more phosphate groups. Nucleotides are monomeric units of a nucleic acid sequence. In RNA, the sugar is a ribose, and in DNA, the sugar is a deoxyribose, i.e. a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. A nucleic acid analog may have any of the phosphate backbone, the sugar, or the nucleobase altered. Examples of nucleic acid analogs include, for example, universal bases or phosphate-sugar backbone analogs, such as peptide nucleic acid (PNA). A “labeled nucleotide” is a nucleotide that has at least an optical label attached thereto. Examples of optical labels include any dye that is capable of emitting an optical signal in response to an excitation wavelength.

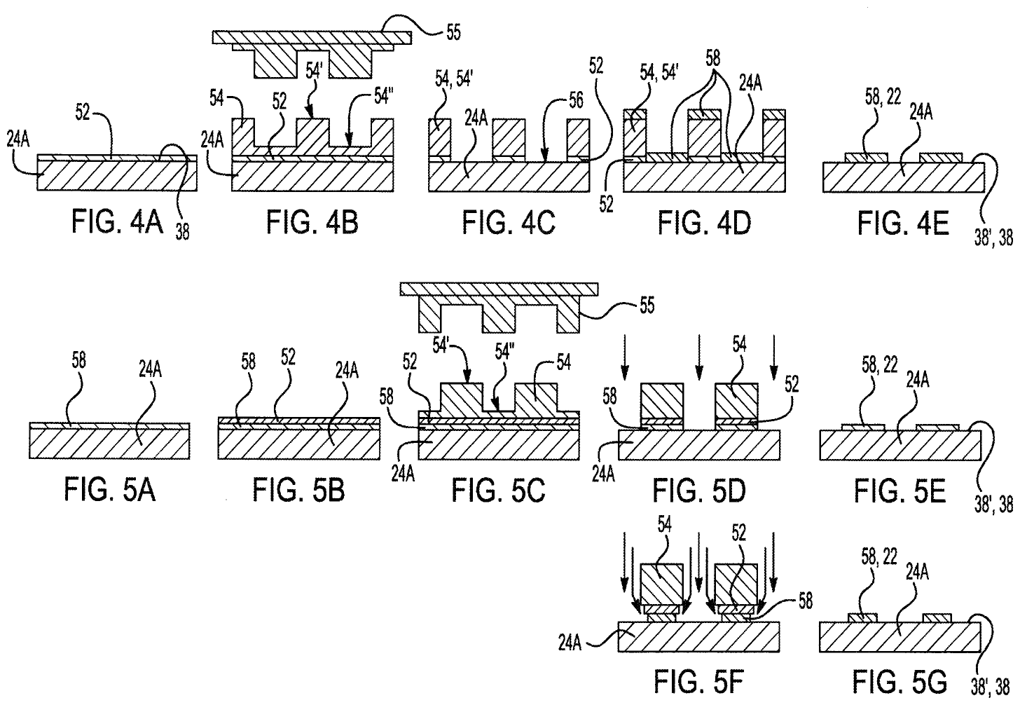

In some examples, the term “over” may mean that one component or material is positioned directly on another component or material. When one is directly on another, the two are in contact with each other. In FIG. 4A, the sacrificial layer 52 may be applied over the substrate 24A so that it is directly on and in contact with the substrate 24A.

In other examples, the term “over” may mean that one component or material is positioned indirectly on another component or material. By indirectly on, it is meant that a gap or an additional component or material may be positioned between the two components or materials. In FIG. 5C, the resin layer 54 is positioned over the substrate 24A such that the two are in indirect contact. More specifically, the resin layer 54 is indirectly on the substrate 24A because the surface chemistry 58 (defining the chemical pads 22) and the sacrificial layer 52 are positioned therebetween.

The terms “particle” and “functionalized particle” are used interchangeably and include i) a core and a hydrogel attached to the core or ii) a hydrogel core, a plurality of primers attached to side chains or arms of the hydrogel or hydrogel core, and a mechanism to attach to a flow cell chemical pad. The particle/functionalized particle enables the formation of a pre-clustered particle.

A “patterned resin” refers to any material that can have protrusions defined therein. Specific examples of resins and techniques for patterning the resins will be described further below.

As used herein, a “positive photoresist” refers to a light sensitive material in which a portion that is exposed to light of particular wavelength(s) becomes soluble to a developer. In these examples, any portion of the positive photoresist exposed to light is at least 95% soluble in the developer. This portion may be referred to herein as a “soluble positive photoresist”. In some examples, the portion of the positive photoresist exposed to light (i.e., the soluble photoresist), is at least 98%, e.g., 99%, 99.5%, 100%, soluble in the developer. With the positive photoresist, the light exposure changes the chemical structure so that the exposed portions of the material become more soluble (than non-exposed portions) in the developer.

In contrast to the soluble positive photoresist, any portion of the positive photoresist not exposed to light is insoluble (less than 5% soluble) in the developer. This portion may be referred to as an “insoluble positive photoresist”. While not soluble in the developer, the insoluble positive photoresist may be at least 99% soluble in a remover that is different from the developer. In some examples, the insoluble positive photoresist is at least 98%, e.g., 99%, 99.5%, 100%, soluble in the remover. The remover may be a solvent or solvent mixture used in a lift-off process.

A “pre-clustered particle” includes i) a core and a hydrogel attached to the core or ii) a hydrogel core, a plurality of primers attached to side chains or arms of the hydrogel or hydrogel core, a plurality of amplicons attached to at least some of the plurality of primers, and a mechanism to attach to a flow cell chemical pad.

Poly(N-(5-azidoacetamidylpentyl)acrylamide-co-acrylamide, referred to herein as “PAZAM,” is one example of the hydrogel or hydrogel core. PAZAM and some other forms of the acrylamide copolymer are represented by the following structure (I):

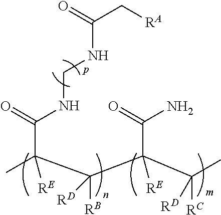

wherein:
R^{A} is selected from the group consisting of azido, optionally substituted amino, optionally substituted alkenyl, optionally substituted alkyne, halogen, optionally substituted hydrazone, optionally substituted hydrazine, carboxyl, hydroxy, optionally substituted tetrazole, optionally substituted tetrazine, nitrile oxide, nitrone, sulfate, and thiol;
R^{B} is H or optionally substituted alkyl;
R^{C}, R^{D}, and R^{E} are each independently selected from the group consisting of H and optionally substituted alkyl;
each of the —(CH₂)ₚ— can be optionally substituted;
p is an integer in the range of 1 to 50;
n is an integer in the range of 1 to 50,000; and
m is an integer in the range of 1 to 100,000.

One of ordinary skill in the art will recognize that the arrangement of the recurring “n” and “m” features in structure (I) are representative, and the monomeric subunits may be present in any order in the polymer structure (e.g., random, block, patterned, or a combination thereof).

The molecular weight of PAZAM and other forms of the acrylamide copolymer may range from about 5 kDa to about 1500 kDa or from about 10 kDa to about 1000 kDa, or may be, in a specific example, about 312 kDa.

In some examples, PAZAM and other forms of the acrylamide copolymer are linear polymers. In some other examples, PAZAM and other forms of the acrylamide copolymer are lightly cross-linked polymers.

In some examples, the gel material may be a variation of structure (I). In one example, the acrylamide unit may be replaced with N,N-dimethylacrylamide

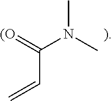

In another example, the acrylamide unit in structure (I) may be replaced with,

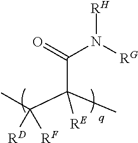

where R^{D}, R^{E}, and R^{F} are each H or a C1-C6 alkyl, and R^{G} and R^{H} are each a C1-C6 alkyl (instead of H as is the case with the acrylamide). In this example, q may be an integer in the range of 1 to 100,000. In another example, the N,N-dimethylacrylamide may be used in addition to the acrylamide unit. In this example, structure (I) may include

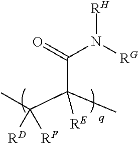

in addition to the recurring “n” and “m” features, where R^{D}, R^{E}, and R^{F} are each H or a C1-C6 alkyl, and R^{G} and R^{H} are each a C1-C6 alkyl. In this example, q may be an integer in the range of 1 to 100,000.

As another example, the recurring “n” feature in structure (I) may be replaced with a monomer including a heterocyclic azido group having structure (II):

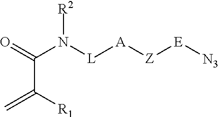

wherein R₁ is H or a C1-C6 alkyl; R² is H or a C1-C6 alkyl; L is a linker including a linear chain with 2 to 20 atoms selected from the group consisting of carbon, oxygen, and nitrogen and 10 optional substituents on the carbon and any nitrogen atoms in the chain; E is a linear chain including 1 to 4 atoms selected from the group consisting of carbon, oxygen and nitrogen, and optional substituents on the carbon and any nitrogen atoms in the chain; A is an N substituted amide with an H or a C1-C4 alkyl attached to the N; and Z is a nitrogen containing heterocycle. Examples of Z include 5 to 10 carbon-containing ring members present as a single cyclic structure or a fused structure. Some specific examples of Z include pyrrolidinyl, pyridinyl, or pyrimidinyl.

As still another example, the hydrogel or hydrogel core material may include a recurring unit of each of structure (III) and (IV):

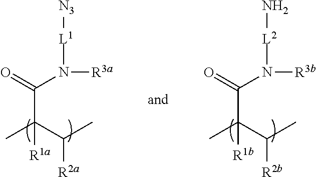

wherein each of R^{1a}, R^{2a}, R^{1b} and R^{2b} is independently selected from hydrogen, an optionally substituted alkyl or optionally substituted phenyl; each of R^{3a} and R^{3b} is independently selected from hydrogen, an optionally substituted alkyl, an optionally substituted phenyl, or an optionally substituted C7-C14 aralkyl; and each L¹ and L² is independently selected from an optionally substituted alkylene linker or an optionally substituted heteroalkylene linker.

As used herein, the term “primer” is defined as a single stranded nucleic acid sequence (e.g., single stranded DNA). Some primers, referred to herein as amplification primers, serve as a starting point for template amplification and cluster generation. Other primers, referred to herein as sequencing primers, serve as a starting point for DNA synthesis. The 5′ terminus of the primer may be modified to allow a coupling reaction with a functional group of the hydrogel. The primer length can be any number of bases long and can include a variety of non-natural nucleotides. In an example, the sequencing primer is a short strand, ranging from 10 to 60 bases, or from 20 to 40 bases.

The term “substrate” refers to a support material upon which surface chemistry for particle attachment is introduced. In some examples of the method, a multi-layer stack is used at the outset and is processed such that one or more layers are removed, and the resulting flow cell includes the flow cell surface chemistry supported by the substrate. In the examples disclosed herein, the substrate has a substantially flat surface. By “substantially flat surface,” it is meant that the substrate does not have convex or concave features defined therein and presents a plane. While the surface may have microscopic or smaller surface roughness, the surface appears smooth and even to the human eye.

“Surface chemistry,” as defined herein, may refer to flow cell surface chemistry or particle surface chemistry. The “flow cell surface chemistry” refers to the chemical makeup of the flow cell chemical pads. The “particle surface chemistry” includes the primers and the mechanism for attaching the particles to the flow cell chemical pads.

The term “tantalum pentoxide” refers to the inorganic compound with the formula Ta₂O₅. This compound is transparent, having a transmittance ranging from about 0.25 (25%) to 1 (100%), to wavelengths ranging from about 0.35 μm (350 nm) to at least 1.8 μm (1800 nm). A “tantalum pentoxide substrate” may comprise, consist essentially of, or consist of Ta₂O₅. In examples where it is desirable for the tantalum pentoxide substrate to transmit electromagnetic energy having any of these wavelengths, the substrate may consist of Ta₂O₅ or may comprise or consist essentially of Ta₂O₅ and other components that will not interfere with the desired transmittance of the substrate.

A “thiol” functional group refers to —SH.

As used herein, the terms “tetrazine” and “tetrazinyl” refer to six-membered heteroaryl group comprising four nitrogen atoms. Tetrazine can be optionally substituted.

“Tetrazole,” as used herein, refer to five-membered heterocyclic group including four nitrogen atoms. Tetrazole can be optionally substituted.

The term “transparent substrate” refers to a material, e.g., in the form of a substrate or layer, that is transparent to a particular wavelength or range of wavelengths. For example, the material may be transparent to wavelength(s) that are used to chemically change a positive or negative photoresist. Transparency may be quantified using transmittance, i.e., the ratio of light energy falling on a body to that transmitted through the body. The transmittance of a transparent substrate or a transparent layer will depend upon the thickness of the substrate or layer and the wavelength of light. In the examples disclosed herein, the transmittance of the transparent substrate or the transparent layer may range from 0.25 (25%) to 1 (100%). The material of the substrate or layer may be a pure material, a material with some impurities, or a mixture of materials, as long as the resulting substrate or layer is capable of the desired transmittance. Additionally, depending upon the transmittance of the substrate or layer, the time for light exposure and/or the output power of the light source may be increased or decreased to deliver a suitable dose of light energy through the transparent substrate and/or layer to achieve the desired effect (e.g., generating a soluble or insoluble photoresist).

### Functionalized and Pre-Clustered Particles

Examples of the functionalized particles 10, 11 are shown in FIG. 1A and examples of the pre-clustered particles 10′, 11′ are shown in FIG. 1B.

Referring specifically to FIG. 1A, two examples of the functionalized particles 10, 11 are depicted. In one example, each of the functionalized particles 11 includes a hydrogel core 12′, a plurality of primers 16A, 16B attached to the hydrogel core 12′, and a flow cell surface attachment mechanism (not shown) to attach to a chemical pad 22 of a flow cell 20 (see FIG. 2B and FIG. 3). In another example, each of the functionalized particles 10 includes a core 12, a hydrogel 14 attached to the core 12, a plurality of primers 16A, 16B attached to side chains or arms of the hydrogel 14, and a flow cell surface attachment mechanism (not shown) to attach to a chemical pad 22 of a flow cell 20.

The functionalized particles 10, 11 may be used in off flow cell amplification techniques, which generate amplicons (also referred to herein as template nucleic acid strands 18) attached to the primers 16A, 16B. Amplification generates pre-clustered particles 10′, 11′, as shown in FIG. 1B. The pre-clustered particles 10′, 11′ are to be used in sequencing.

### Core

As mentioned, some examples of the functionalized particle 10 include the core 12. The core 12 is generally rigid and is insoluble in an aqueous liquid. The core 12 may also be inert to the surface chemistry that is attached to the hydrogel 14 that coats the core 12. For example, the core 12 can be inert to chemistry used to attach the primer(s) 16A, 16B, used in sequencing reactions, etc. Examples of suitable materials for the core 12 include inert and/or magnetic particles (e.g., magnetic FeOₓ, silica coated FeOₓ), plastics (e.g., polytetrafluoroethylene (PTFE), some polyacrylics, polypropylene, polyethylene, polybutylene, polyurethanes, polystyrene and other styrene copolymers, nylon (i.e., polyamide), polycaprolactone (PCL), nitrocellulose, silica (SiO₂), silica-based materials (e.g., functionalized SiO₂), carbon, or metals.

### Hydrogel or Hydrogel Core

As noted herein, in some examples, the core 12 may be coated with a hydrogel material (e.g., hydrogel 14), and in other examples, the hydrogel core 12′ is made up of the hydrogel material. In either example, the hydrogel material is a polymeric hydrogel. The polymeric hydrogel refers to a semi-rigid polymer that is permeable to liquids and gases. The polymeric hydrogel can swell when liquid (e.g., water) is taken up and that can contract when liquid is removed, e.g., by drying. While a hydrogel material may absorb water, it is not water-soluble.

In some examples, the polymeric hydrogel is in the form of a hydrogel core 12′ or is a hydrogel 14 coated on the core 12. Methods for forming the hydrogel core 12′ and for coating the polymeric hydrogel 14 on the core 12 are described in more detail below.

In some examples, the polymeric hydrogel material is poly(N-(5 -azidoacetamidylpentyl)acrylamide-co-acrylamide (PAZAM) or another of the acrylamide copolymers disclosed herein, poly(ethylene glycol) (PEG)-acrylate, PEG-diacrylate, PEG-amine, PEG-carboxylate, PEG-dithiol, PEG-epoxide, PEG-isocyanate, PEG-maleimide, crosslinked poly(methyl methacrylate) (PMMA), polyvinylpyrrolidone (PVPON), polyvinyl alcohol (PVA), polyethylene oxide-polypropylene oxide block copolymers (PEO-PPO), poly(hydroxyethyl methacrylate) (PHEMA), poly(N-isopropylacrylamide) (PNIPAAm), poly(lactic acid) -poly(ethylene glycol) block copolymers, poly(ethylene glycol)-poly(lactic-co-glycolic acid) block copolymers, poly(acrylic-co-vinylsulfonic acid), poly(acrylamide-co -vinylsulfonic acid), poly(L-aspartic acid), poly(aspartamide), adipic dihydrazide modified or aldehyde modified poly(L-glutamic acid), bisacrylamide, or hydrogels based on one or more of polylysine, starch, agar, agarose, heparin, alginate, alginate sulfate, dextran sulfate, hyaluronan, pectin, carrageenan, gelatin, chitosan, cellulose, and collagen, or combinations or mixtures thereof.

In some examples, the polymeric hydrogel 14 or the hydrogel core 12′ is PAZAM or another acrylamide based copolymer material. In some examples, the polymeric hydrogel 14 or the hydrogel core 12′ is an alginate, acrylamide, or a PEG based material. In some examples, the polymeric hydrogel 14 or the hydrogel core 12′ is a PEG based material with acrylate-dithiol, or epoxide-amine reaction chemistries. In some examples, the polymeric hydrogel 14 forms a polymer shell that includes PEG-maleimide/dithiol oil, PEG-epoxide/amine oil, PEG-epoxide/PEG-amine, or PEG-dithiol/PEG-acrylate.

Polymeric hydrogels 14 or the hydrogel core 12′ may be prepared by cross-linking hydrophilic biopolymers or synthetic polymers or polymerizing suitable monomers and then cross-linking the resulting polymer. Thus, in some examples, the hydrogel 14 or the hydrogel core 12′ may include a crosslinker. As used herein, the term “crosslinker” refers to a molecule that can form a three-dimensional network when reacted with the appropriate base monomers. Examples of the previously listed hydrogel polymers may include one or more crosslinkers, such as N,N′-bis(acryloyl)cystamine, diamines, dopamine, cysteamine, and aminosilanes. In some examples, a crosslinker forms a disulfide bond in the hydrogel polymer, thereby linking hydrogel polymers.

Methods for polymerizing the polymeric hydrogel 14 or the hydrogel core 12′ and/or polymerizing the polymeric hydrogel 14 from the core 12 are described in more detail below.

In some of the examples disclosed herein, the polymeric hydrogel 14 or the hydrogel core 12′ is a copolymer including at least one acrylamide monomer unit, and is a linear polymeric hydrogel or branched polymeric hydrogel (e.g., a dendrimer).

The linear or branched polymeric hydrogel 14 or hydrogel core 12′ may include a first recurring unit of formula (I):

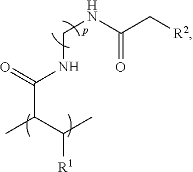

wherein:
R¹ is selected from the group consisting of —H, a halogen, an alkyl, an alkoxy, an alkenyl, an alkynyl, a cycloalkyl, an aryl, a heteroaryl, a heterocycle, and optionally substituted variants thereof;
R² is selected from the group consisting of an azido, an optionally substituted amino, an optionally substituted alkenyl, an optionally substituted alkyne, a halogen, an optionally substituted hydrazone, an optionally substituted hydrazine, a carboxyl, a hydroxy, an optionally substituted tetrazole, an optionally substituted tetrazine, nitrile oxide, nitrone, sulfate, and thiol; each (CH₂)ₚ can be optionally substituted; and p is an integer from 1 to 50; a second recurring unit of formula (II):

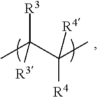

wherein: each of R³, R^{3′}, R⁴, R^{4′} is independently selected from the group consisting of —H, R⁵, —OR⁵, —C(O)OR⁵, —C(O)R⁵, —OC(O)R5, —C(O)NR⁶R⁷, and —NR⁶R⁷; R⁵ is selected from the group consisting of —H, —OH, an alkyl, a cycloalkyl, a hydroxyalkyl, an aryl, a heteroaryl, a heterocycle, and optionally substituted variants thereof; and each of R⁶ and R⁷ is independently selected from the group consisting of —H and an alkyl.

In an example of the polymeric hydrogel 14 or hydrogel core 12′, R¹ is —H; R² is an azido; each of R^{3′}, R⁴, and R^{4′} is —H; R³ is —C(O)NR⁶R⁷, where each of R⁶ and R⁷ is —H; and p is 5. This polymeric hydrogel 14 or hydrogel core 12′ is poly(N-(5-azidoacetamidylpentyl)acrylamide-co-acrylamide, or PAZAM. In a variation of PAZAM, R¹ is —H; R² is an azido; each of R^{3′}, R⁴, and R^{4′} is —H; R³ is —C(O)NR⁶R⁷, where each of R⁶ and R⁷ is a C1-C6 alkyl (e.g., —CH₃); and p is 5.

In some examples, R² of some of the recurring units of formula (I) is replaced with tetramethylethylenediamine (TeMED). TeMED is a reaction promoter that may be introduced during copolymerization. As a result of a side reaction, TeMED replaces some of the azide (N₃) or other R² groups. While this reaction reduces the azide (or other R² examples) content of the copolymer chains, it also introduces a branching site. The branching sites may provide a location where the copolymer chains can branch to one other.

In other examples, a third recurring unit of formula (II) may be included, with the caveat that the second and third recurring units are different. For example, in the second recurring unit each of R^{3′}, R⁴, and R^{4′} is —H; R³ is —C(O)NR⁶R⁷, where each of R⁶ and R⁷ is —H, and in the third recurring unit, each of R^{3′}, R⁴, and R^{4′} is —H; R³ is —C(O)NR⁶R⁷, where each of R⁶ and R⁷ is a C1-C6 alkyl.

The number of first recurring units (formula (I)) may be an integer ranging from 2 to 50,000, and the number of second recurring units (formula (II)) may be an integer ranging from 2 to 100,000. When the third recurring unit is included, the number of units may be an integer in the range of 1 to 100,000. It is to be understood that the incorporation of the individual units may be statistical, random, or in block, and may depend upon the method used to synthesize the polymeric hydrogel 14.

In other examples of the polymeric hydrogel 14 or hydrogel core 12′, the first recurring unit of formula (I) may be replaced with a heterocyclic azido group of formula (III):

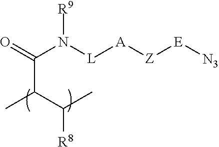

wherein R⁸ is H or a C1-C6 alkyl; R⁹ is H or a C1-C6 alkyl; L is a linker including a linear chain with 2 to 20 atoms selected from the group consisting of carbon, oxygen, and nitrogen and 10 optional substituents on the carbon and any nitrogen atoms in the chain; E is a linear chain including 1 to 4 atoms selected from the group consisting of carbon, oxygen and nitrogen, and optional substituents on the carbon and any nitrogen atoms in the chain; A is an N substituted amide with an H or a C1-C4 alkyl attached to the N; and Z is a nitrogen containing heterocycle. Examples of Z include 5 to 10 carbon-containing ring members present as a single cyclic structure or a fused structure. Some specific examples of Z include pyrrolidinyl, pyridinyl, or pyrimidinyl.

In one example, formula (III) is the first recurring unit and formula (II) is the second recurring unit. In another example, formula (III) is the first recurring unit, one example of formula (II) is the second recurring unit, and a different example of formula (III) is the third recurring unit.

It is to be understood that other hydrogel materials may be used for the hydrogel 14, as long as they are functionalized to graft oligonucleotide primers 16A, 16B thereto and are capable of attaching to the core 12. It is also to be understood that other hydrogel materials may be used for the hydrogel core 12′, as long as they are functionalized to graft oligonucleotide primers 16A, 16B thereto.

Some examples of other suitable polymeric materials for the hydrogel 14 or hydrogel core 12′ include functionalized polysilanes, such as norbornene silane, azido silane, alkyne functionalized silane, amine functionalized silane, maleimide silane, or any other polysilane having functional groups that can attach the oligonucleotide primers 16A, 16B. Other examples of suitable hydrogel materials for the hydrogel 14 or the hydrogel core 12′ include those having a colloidal structure, such as agarose; or a polymer mesh structure, such as gelatin; or a cross-linked polymer structure, such as polyacrylamide polymers and copolymers, silane free acrylamide (SFA), or an azidolyzed version of SFA. Examples of suitable polyacrylamide polymers may be synthesized from acrylamide and an acrylic acid or an acrylic acid containing a vinyl group, or from monomers that form [2+2] photo-cycloaddition reactions. Still other examples of suitable polymeric hydrogel materials include mixed copolymers of acrylamides and acrylates. A variety of polymer architectures containing acrylic monomers (e.g., acrylamides, acrylates etc.) may be utilized in the examples disclosed herein, such as highly branched polymers, including dendrimers. For example, the monomers (e.g., acrylamide, etc.) may be incorporated, either randomly or in block, into the branches (arms) of a dendrimer.

An example of the dendrimeric polymeric hydrogel material includes a dendritic core with recurring units of formulas (II) and (III) in the arms extending from the core. The dendritic core may have anywhere from 3 arms to 30 arms.

The dendritic core may be any multi-functional component that enables a controlled polymerization mechanism, which leads to a defined arm length in the polymer structure and an at least substantially uniform arm length between polymer structures. In an example, the arms of the dendritic core are identical to each other.

The central molecule/compound of the dendritic core may be any multi-functional molecule, such as macrocycles (e.g., cyclodextrins, porphyrins, etc.), extended pi-systems (e.g., perylenes, fullerenes, etc.), metal-ligand complexes, polymeric cores, etc. Some specific examples of the central molecule/compound of the dendritic core include a phenyl group, benzoic acid, pentraerythritol, a phosphazene group, etc.

The dendritic core includes arms that extend from the central molecule/compound. Each arm may include a group that enables the monomers of formula (II) and (III) to be incorporated. In one example, a thiocarbonylthio group is included in each arm, and thus includes a reversible addition-fragmentation chain transfer agent (a RAFT agent). In another example, the dendritic core includes an atom transfer radical polymerization (ATRP) initiator in each arm. In still another example, the dendritic core includes a nitroxide (aminooxyl) mediated polymerization (NMP) initiator in each arm.

It is to be understood that functional groups in one or more of the recurring units of the hydrogel material of the hydrogel 14 or the hydrogel core 12′ are capable of attaching the primers 16A, 16B. These functional groups (e.g., R² in formula (I), NH₂, N₃, etc.) may be located in the side chains of the linear or branched polymeric hydrogel material. As noted, one example of the branched polymeric hydrogel is a dendrimer, and in an example, the primer-grafting functional groups are located in each of the arms of the dendrimer. These functional groups may be introduced as part of the monomer(s) used in copolymerization. To control the number of primer 16A, 16B anchorage points, the monomer bearing the functional group may be increased or decreased. These functional groups may alternatively be introduced after copolymerization.

In the functionalized particle 10, the thickness of the hydrogel 14 on the core 12 ranges from about 10 nm to about 200 nm. The hydrogel 14 can be in a dry state or can be in a swollen state, where it uptakes liquid. The 10 nm thickness represents the hydrogel 14 in the fully dry state, and the 200 nm thickness represents the hydrogel 14 in the fully swollen state.

The weight average molecular weight of hydrogel material used for the hydrogel 14 or the hydrogel core 12′ (linear or branched) ranges from about 10 kDa to about 2,000 kDa. In other examples, the weight average molecular weight ranges from about 100 kDa to about 400 kDa. Increasing the molecular weight will increase the thickness of the coating of the hydrogel 14. For the dendrimer version of the hydrogel 14, the branching number may also be used to achieve the desired thickness. Increasing the branching number will also increase the thickness of the coating. In an example, the branching number ranges from 3 to 30.

### Primer Set

The functionalized particles 10, 11 also include the primers 16A, 16B. The polymeric hydrogel 14 and/or the hydrogel core 12′ provides a surface for attachment of the primers 16A, 16B and the flow cell surface attachment mechanism (not shown).

The primer set attached to the polymeric hydrogel 14 or the hydrogel core 14′ includes two different primers 16A, 16B that are used in sequential paired end sequencing. As examples, the primer set may include P5 and P7 primers, P15 and P7 primers, or any combination of the PA primers, the PB primers, the PC primers, and the PD primers set forth herein. As examples, the primer set may include any two PA, PB, PC, and PD primers, or any combination of one PA primers and one PB, PC, or PD primer, or any combination of one PB primer and one PC or PD primer, or any combination of one PC primer and one PD primer. Examples of P5 and P7 primers are used on the surface of commercial flow cells sold by Illumina Inc. for sequencing, for example, on HiSeq™, HiSeqX™, MiSeq™, MiSegDX™, MiNISeq™, NextSeq™, NextSegDX™, NovaSeq™, iSEQ™, Genome Analyzer™, and other instrument platforms. The P5 and P7 primers have a universal sequence for seeding and/or amplification purposes.

The P5 primer is:

P5: 5′ → 3′
(SEQ. ID. NO. 1)
AATGATACGGCGACCACCGAGAUCTACAC

The P7 primer may be any of the following:

P7 #1: 5′ → 3′
(SEQ. ID. NO. 2)
CAAGCAGAAGACGGCATACGAnAT
P7 #2: 5′ → 3′
(SEQ. ID. NO. 3)
CAAGCAGAAGACGGCATACnAGAT
P7 #3: 5′ → 3′
(SEQ. ID. NO. 4)
CAAGCAGAAGACGGCATACnAnAT

where “n” is 8-oxoguanine in each of the sequences.

The P15 primer is:

P15: 5′ → 3′
(SEQ. ID. NO. 5)
AATGATACGGCGACCACCGAGAnCTACAC

where “n” is allyl-T.

The other primers (PA-PD) mentioned above include:

PA 5′ → 3′
(SEQ. ID. NO. 6)
GCTGGCACGTCCGAACGCTTCGTTAATCCGTTGAG
cPA (PA′) 5′ → 3′
(SEQ. ID. NO. 7)
CTCAACGGATTAACGAAGCGTTCGGACGTGCCAGC
PB 5′ → 3′
(SEQ. ID. NO.8)
CGTCGTCTGCCATGGCGCTTCGGTGGATATGAACT
cPB (PB′) 5′ → 3′
(SEQ. ID. NO. 9)
AGTTCATATCCACCGAAGCGCCATGGCAGACGACG
PC 5′ → 3′
(SEQ. ID. NO. 10)
ACGGCCGCTAATATCAACGCGTCGAATCCGCAACT
cPC (PC′) 5′ → 3′
(SEQ. ID. NO. 11)
AGTTGCGGATTCGACGCGTTGATATTAGCGGCCGT
PD 5′ → 3′
(SEQ. ID. NO. 12)
GCCGCGTTACGTTAGCCGGACTATTCGATGCAGC
cPD (PD′) 5′ → 3′
(SEQ. ID. NO. 13)
GCTGCATCGAATAGTCCGGCTAACGTAACGCGGC

While not shown in the example sequences for PA-PD, it is to be understood that any of these primers may include a cleavage site, such as uracil, 8-oxoguanine, allyl-T, etc. at any point in the strand.

It is to be understood that the cleavage sites of the primers 16A, 16B in the primer set are orthogonal to each other (i.e., one cleavage site is not susceptible to the cleaving agent used for the other cleavage site), so that after amplification, forward or reverse strands can be cleaved, leaving the other of the reverse or forward strands for sequencing.

Each of the primers 16A, 16B disclosed herein may also include a polyT sequence at the 5′ end of the primer sequence. In some examples, the polyT region includes from 2 T bases to 20 T bases. As specific examples, the polyT region may include 3, 4, 5, 6, 7, or 10 T bases.

The 5′ end of each primer 16A, 16B may also include a linker. Any linker that includes a terminal alkyne group or another suitable terminal functional group that can attach to the surface functional groups of the hydrogel 14 or the hydrogel core 12′ may be used. In one example, the primers 16A, 16B are 5′ terminated with hexynyl.

The immobilization of the primers 16A, 16B may be by single point covalent attachment at the 5′ end of the primers 16A, 16B. The attachment will depend, in part, on the functional groups of the hydrogel 14 or the hydrogel core 12′. Examples of terminated primers that may be used include an alkyne terminated primer, a tetrazine terminated primer, an azido terminated primer, an amino terminated primer, an epoxy or glycidyl terminated primer, a thiophosphate terminated primer, a thiol terminated primer, an aldehyde terminated primer, a hydrazine terminated primer, a phosphoramidite terminated primer, and a triazolinedione terminated primer. In some specific examples, a succinimidyl (NHS) ester terminated primer may be reacted with an amine of the hydrogel 14 or the hydrogel core 12′, an aldehyde terminated primer may be reacted with a hydrazine of the hydrogel 14 or the hydrogel core 12′, or an alkyne terminated primer may be reacted with an azide of the hydrogel 14 or the hydrogel core 12′, or an azide terminated primer may be reacted with an alkyne or DBCO (dibenzocyclooctyne) of the hydrogel 14 or the hydrogel core 12′, or an amino terminated primer may be reacted with an activated carboxylate group or NHS ester of the hydrogel 14 or the hydrogel core 12′, or a thiol terminated primer may be reacted with an alkylating reactant (e.g., iodoacetamine or maleimide) of the hydrogel 14 or the hydrogel core 12′, or a phosphoramidite terminated primer may be reacted with a thioether of the hydrogel 14 or the hydrogel core 12′. While several examples have been provided, it is to be understood that a functional group that can be attached to the primer 16A, 16B and that can attach to a functional group of the hydrogel 14 or the hydrogel core 12′ may be used.

### Flow Cell Surface Attachment Mechanism

The functionalized particles 10, 11 and the pre-clustered particles 10′, 11′ formed therefrom are also capable of anchoring to a chemical pad 22 on a flow cell substrate 24A, 24B. As such, the functionalized particles 10, 11 and the pre-clustered particles 10′, 11′ include a flow cell surface attachment mechanism (not shown) that is capable of binding, attaching, or attracting (e.g., electrostatically, etc.) to the flow cell chemical pad 22. In the examples disclosed herein, the flow cell surface attachment mechanism is selected from the group consisting of a capture primer, an alkene, an alkyne, biotin, and a charged polymer.

In some examples, the flow cell surface attachment mechanism is a component of the functionalized particles 10, 11, and thus of the pre-clustered particles 10′, 11′, that enables them to be anchored without further functionalization. For example, when the hydrogel 14 or hydrogel core 12′ includes an alkene or an alkyne functional group, these functional groups serve as the flow cell surface attachment mechanism. In these instances, the pre-clustered particles 10′, 11′ may be anchored to the flow cell chemical pads 22 via click chemistry. As an example, the hydrogel 14 or hydrogel core 12′ may be functionalized with an alkene (e.g., vinyl-PEG) or an alkyne (e.g., dibenzocyclooctyne), and the flow cell chemical pad 22 may include an azide that can attach to the alkene or alkyne of the pre-clustered particle 10′, 11′. In one example, the chemical pads 22 may be poly(N-(5-azidoacetamidylpentyl)acrylamide-co-acrylamide (PAZAM) or other acrylamide with azide surface groups which can attach to the pre-clustered particles 10′, 11′ when the hydrogel 14 includes an alkene or an alkyne functional group as the flow cell surface attachment mechanism.

For another example, when the hydrogel 14 or the hydrogel core 12′ is formed of a charged polymer, it includes a charged or chargeable functional group as the flow cell surface attachment mechanism. In this example, the pre-clustered particles 10′, 11′ may be anchored to the flow cell chemical pads 22 via electrostatic forces because the chemical pads 22 include a counter ion of the charged polymer. In one example, the charged polymer is selected from the group consisting of polylysine, polyethyleneimine, and a polypeptide; and the counter ion is selected from the group consisting of an oligonucleotide, polyacrylic acid, and polystyrene sulfonate.

In other examples, the flow cell surface attachment mechanism is one of the primers 16A, 16B of the primer set attached to the surface of the pre-clustered particles 10′, 11′ that enables it to be anchored on the flow cell chemical pads 22. In this example, the 3′ end of one or some of the primers 16A, 16B includes a functional group that can attach to the flow cell chemical pad 22. As one example, the chemical pads 22 are a modified PAZAM with amine groups rather than azide groups. In this example, the PAZAM pads are treated with an azide reducing agent (e.g., phosphine, tin(IV) 1,2-benzenedithiolate in the presence of NaBH₄, dichloroindium hydride, borontrifluoride diethyl etherate and sodium iodide, and copper nanoparticles in water in the presence of ammonium formate) to generate amine groups. One or more of the primers 16A, 16B of the pre-clustered particles 10′, 11′ have a 3′ terminal group (e.g., negatively charged DNA or succinimidyl (NHS) ester) that can react with the amine groups of these flow cell chemical pads 22. The attachment mechanism between the primers 16A, 16B and the chemical pads 22 may involve one or more of electrostatic forces, hydrogen bonding, and/or covalent bonding.

In other examples, the flow cell surface attachment mechanism is added to the functionalized particle 10, 11, and thus the pre-clustered particle 10′, 11′, that enables it to be anchored on the flow cell chemical pads 22. As one example, a capture primer may be grafted to the hydrogel 14 or the hydrogel core 12′ that is complementary to a target primer on the flow cell chemical pads 22. As one example, the flow cell chemical pads 22 may include a PX primer for capturing a cPX primer attached to the pre-clustered particle 10′, 11′. The density of the PX motifs on each chemical pad 22 should be relatively low in order to minimize the attachment of multiple pre-clustered particles 10′, 11′ at each chemical pad 22. The PX primer may be:

PX 5′ → 3′
(SEQ. ID. NO. 14)
AGGAGGAGGAGGAGGAGGAGGAGG

The pre-clustered particle 10′, 11′ includes the capture primer that is partially complementary to the PX primer, which in this particular example is:

cPX (PX′) 5′ → 3′
(SEQ. ID. NO. 15)
CCTCCTCCTCCTCCTCCTCCTCCT

In other examples, the flow cell surface attachment mechanism is a member of a receptor-ligand binding pair (e.g., streptavidin, biotin, etc.) that is capable of binding to the flow cell chemical pad 22, which includes the other member of the binding pair. As one example, the hydrogel 14 or hydrogel core 12′ may be functionalized to non-covalently attach to the flow cell chemical pad 22. More specifically, the hydrogel 14 or hydrogel core 12′ may be functionalized with a first member of a binding pair, which interacts with a second member of the binding pair that is attached to the flow cell chemical pad 22. In one example binding pair, the first member and the second member respectively include streptavidin and biotin. In one example, the functionalized particle 10, 11, and thus the pre-clustered particle 10′, 11′, is biotinylated. Biotin-alkyne is attached to the surface of the polymeric hydrogel 14 or hydrogel core 12′ of the pre-clustered particle 10′, 11′ through some of the surface groups, such as the R^{A} groups of structure (I) (i.e., the azide, tetrazine, or other functional group that can attach to the alkyne). The biotin is attached to an alkyne linker, such as bicyclo[6.1.0]nonyne (BCN), which can covalently attach to some of the R^{A} groups or other surface groups. In this particular example, the flow cell chemical pads 22 may be formed of streptavidin or the streptavidin may be a functional group connected to the flow cell chemical pad 22.

As other examples, a functional group for covalent attachment to the chemical pad 22 or a member of a binding pair may be introduced to one of the monomers used in polymerization or the hydrogel 14 or the hydrogel core 12′, or may be grafted to the hydrogel 14 or the hydrogel core 12′ after polymerization, or may be chemically introduced to the hydrogel 14 or the hydrogel core 12′ after polymerization. Any of the mechanisms described herein may be used for attaching the pre-clustered particles 10′, 11′ to the flow cell chemical pads 22, and will depend on the particular chemistry of the flow cell chemical pad 22.

### Methods for Making the Functionalized Particles

To make the functionalized particle 10 shown in FIG. 1A, the hydrogel 14 is coated on the core 12. The hydrogel material may be coated on the core 12 using any suitable deposition techniques. Examples of suitable deposition techniques include dip coating, dunk coating, spin coating, spray coating, puddle dispensing, ultrasonic spray coating, etc. In an example, the core 12 may be suspended in the polymeric hydrogel material and exposed to conditions (e.g., heat) that will initiate the attachment of the polymeric hydrogel 14 to the core 12. The type of attachment that is formed will depend upon the chemistry of the hydrogel 14 and the core 12.

Prior to forming the functionalized particle 10, the hydrogel material may be prepared by polymerizing the monomer(s) that are to form the hydrogel. The polymerization process and process conditions will depend upon the monomer(s). In an example, the hydrogel 14 may be synthesized using reversible addition-fragmentation chain transfer (RAFT) polymerization. While RAFT polymerization may be used, it is to be understood that other polymerization processes may also be used. Other suitable polymerization processes include atom transfer radical polymerization (ATRP), nitroxide mediated radical (NMP) polymerization in combination with RAFT or ATRP, NMP with an additional cross-linking step, cobalt-mediated polymerization, group transfer polymerization (GTP), ring opening polymerization (ROP), ionic polymerization, or any other polymerization process that either directly or indirectly yields the desired linear or branched architecture.

Once the hydrogel material is formed and coated on the core 12 to form the hydrogel 14, the primers 16A, 16B may be grafted to the hydrogel 14. Grafting may involve dunk coating, which involves immersing the hydrogel 14 in a primer solution or mixture, which may include the primer(s) 16A, 16B, water, a buffer, and a catalyst. Other grafting techniques may involve spray coating, puddle dispensing, or another suitable method that will attach the primer(s) 16A, 16B to the hydrogel 14. With any of the grafting methods, the primers 16A, 16B react with reactive groups of the hydrogel 14.

In other examples, the primers 16A, 16B are grafted to the hydrogel material before it is coated on the core 12. In this example, the core 12 may be suspended in the pre-grafted polymeric hydrogel material and exposed to conditions (e.g., heat) that will initiate the attachment of the pre-grafted polymeric hydrogel 14 to the core 12. In these examples, additional grafting is not performed.

To make the functionalized particle 11 shown in FIG. 1A, the primers 16A, 16B are grafted to the hydrogel core 12′. The hydrogel core 12′ may be formed by emulsion polymerizing the monomer(s) in the presence of seed latexes and a surfactant, the latter of which promotes the coagulation of particles. Particle growth depends on the nucleation speed, and can be controlled by adjusting the monomer ratio, the conversion rate, the polymerization temperature, etc. Any of the grafting techniques disclosed herein may be used to attach the primers 16A, 16B to the hydrogel core 12′.

Some examples of the methods for making the functionalized particle 10 or 11 may also include attaching the surface attachment mechanism. When the surface attachment mechanism is part of the one or more of the primers 16A, 16B (e.g., a 3′ terminal group that can react with an amine of the chemical pad 22) or part of the hydrogel 14 or hydrogel core 12′ (e.g., an alkene, an alkyne, or a charged polymer), no additional processes are performed to introduce the surface attachment mechanism.

When the surface attachment mechanism is the capture primer, the capture primer may be grafted to the hydrogel 14 or hydrogel core 12′ using any of the grafting techniques disclosed herein.

### Methods for Making the Pre-Clustered Particles

The functionalized particles 10, 11 may be used in an off flow cell amplification process for the generation of template nucleic acid strands 18 (FIG. 1B) that are attached to the hydrogel 14 or hydrogel core 12′. This forms the pre-clustered particles 10′, 11′, which can then be used in sequencing.

At the outset of template strand formation, library templates may be prepared from any nucleic acid sample (e.g., a DNA sample or an RNA sample). The DNA nucleic acid sample may be fragmented into single-stranded, similarly sized (e.g., <1000 bp) DNA fragments. The RNA nucleic acid sample may be used to synthesize complementary DNA (cDNA), and the cDNA may be fragmented into single-stranded, similarly sized (e.g., <1000 bp) cDNA fragments. During preparation, adapters may be added to the ends of any of the fragments. Through reduced cycle amplification, different motifs may be introduced in the adapters, such as sequencing primer binding sites, indices, and regions that are complementary to the primers 16A, 16B on the functionalized particles 10, 11. In some examples, the fragments from a single nucleic acid sample have the same adapters added thereto. The final library templates include the DNA or cDNA fragment and adapters at both ends. The DNA or cDNA fragment represents the portion of the final library template that is to be sequenced.

A plurality of library templates may be introduced to a suspension containing the functionalized particles 10, 11, which includes the liquid carrier and the functionalized particles 10, 11. Multiple library templates are hybridized, for example, to one of two types of primers of the primer set 16A, 16B, which are immobilized to the functionalized particles 10, 11.

Amplification of the template nucleic acid strand(s) on the functionalized particles 10, 11 may be initiated to form a cluster of the template strands 18 across the particle surface. This generates pre-clustered particles 10′, 11′. In one example, amplification involves cluster generation. In one example of cluster generation, the library templates are copied from the hybridized primers by 3′ extension using a high-fidelity DNA polymerase. The original library templates are denatured, leaving the copies immobilized all around the functionalized particles 10, 11. Isothermal bridge amplification or some other form of amplification may be used to amplify the immobilized copies. For example, the copied templates loop over to hybridize to an adjacent, complementary primer, and a polymerase copies the copied templates to form double stranded bridges, which are denatured to form two single stranded strands. These two strands loop over and hybridize to adjacent, complementary primers and are extended again to form two new double stranded loops. The process is repeated on each template copy by cycles of isothermal denaturation and amplification to create dense clonal clusters on the functionalized particles 10, 11. Each cluster of double stranded bridges is denatured. In an example, the reverse strand is removed by cleaving at the cleavage site (e.g., specific base cleavage), leaving forward template strands. In another example, the forward strand is removed by cleaving at the cleavage site, leaving reverse template strands. Clustering results in the formation of the pre-clustered particles 10′, 11′, which includes several template strands 18 immobilized on the functionalized particles 10, 11. This example of clustering is referred to as bridge amplification, and is one example of the amplification that may be performed. It is to be understood that other amplification techniques may be used, e.g., exclusion amplification.

The pre-clustered particles 10′, 11′ may be washed to remove unreacted library templates, etc. and suspended in a fresh carrier liquid.

### Flow Cells for Use With the Pre-Clustered Particles

The pre-clustered particles 10′, 11′ may be used with any flow cell 20 (FIG. 2A) that includes flow cell chemical pads 22 (FIG. 2B). An example of the flow cell 20 is depicted from the top view in FIG. 2A, and an example of the flow cell architecture, including flow cell chemical pads 22, is shown in as a perspective view in FIG. 2B and as a cross-sectional view in FIG. 3.

As will be discussed in reference to FIG. 3, an example of the flow cell 20 include two opposed substrates 24A, 24B, each of which is configured with flow cell chemical pads 22. In these examples, a flow channel 26 is defined between the two opposed substrates 24A, 24B. In other examples, the flow cell 20 includes one substrate 24A configured with flow cell chemical pads 22 and a lid attached to the substrate 24A. In these examples, the flow channel 26 is defined between the substrate 24A and the lid.

In the example shown in FIG. 3, the substrates 24A, 24B are single layered structures. Examples of suitable single layered structures for the substrate 24A, 24B include epoxy siloxane, glass, modified or functionalized glass, plastics (including acrylics, polystyrene and copolymers of styrene and other materials, polypropylene, polyethylene, polybutylene, polyurethanes, polytetrafluoroethylene (such as TEFLON® from Chemours), cyclic olefins/cyclo-olefin polymers (COP) (such as ZEONOR® from Zeon), polyimides, etc.), nylon (polyamides), ceramics/ceramic oxides, silica, fused silica, or silica-based materials, aluminum silicate, silicon and modified silicon (e.g., boron doped p+ silicon), silicon nitride (Si₃N₄), silicon oxide (SiO₂), tantalum pentoxide (Ta₂O₅) or other tantalum oxide(s) (TaOₓ), hafnium oxide (HfO₂), carbon, metals, inorganic glasses, or the like.

In an example, the substrates 24A, 24B may be round and have a diameter ranging from about 2 mm to about 300 mm, or may be a rectangular sheet or panel having its largest dimension up to about 10 feet (˜3 meters). In an example, the substrate 24A, 24B is a wafer having a diameter ranging from about 200 mm to about 300 mm. Wafers may subsequently be diced to form an individual flow cell substrate. In another example, the substrate 24A, 24B is a die having a width ranging from about 0.1 mm to about 10 mm. While example dimensions have been provided, it is to be understood that a substrate 24A, 24B with any suitable dimensions may be used. For another example, a panel may be used that is a rectangular substrate, which has a greater surface area than a 300 mm round wafer. Panels may subsequently be diced to form individual flow cells.

The flow cell 20 also includes the flow channel 26. While several flow channels 26 are shown in FIG. 2A, it is to be understood that any number of channels 26 may be included in the flow cell 20 (e.g., a single channel 26, four channels 26, etc.). Each flow channel 26 may be isolated from each other flow channel 26 in a flow cell 20 so that fluid introduced into any particular flow channel 26 does not flow into any adjacent flow channel 26.

A portion of the flow channel 26 may be defined in the substrate 24A, 24B, using any suitable technique that depends, in part, upon the material(s) of the substrate 24A, 24B. In one example, a portion of the flow channel 26 is etched into a glass substrate, such as substrate 24A, 24B.

In an example, the flow channel 26 has a substantially rectangular configuration with rounded ends. The length and width of the flow channel 26 may be smaller, respectively, than the length and width of the substrate 24A, 24B so that a portion of the substrate surface surrounding the flow channel 26 is available for attachment to another substrate 24A, 24B or to a lid. In some instances, the width of each flow channel 26 can be at least about 1 mm, at least about 2.5 mm, at least about 5 mm, at least about 7 mm, at least about 10 mm, or more. In some instances, the length of each flow channel 26 can be at least about 10 mm, at least about 25 mm, at least about 50 mm, at least about 100 mm, or more. The width and/or length of each flow channel 26 can be greater than, less than or between the values specified above. In another example, the flow channel 26 is square (e.g., 10 mm×10 mm).

The depth of each flow channel 26 can be as small as a few monolayers thick, for example, when microcontact, aerosol, or inkjet printing is used to deposit the separate material 36 that defines the flow channel walls and that at least partially separated one flow channel 26 from an adjacent flow channel 26. In other examples, the depth of each flow channel 26 can be about 1 μm, about 10 μm, about 50 μm, about 100 μm, or more. In an example, the depth may range from about 10 μm to about 100 μm. In another example, the depth is about 5 μm or less. It is to be understood that the depth of each flow channel 26 can also be greater than, less than or between the values specified above.

In the example shown in FIG. 3, the substrate 24A, 24B has a substantially flat surface 38; and the plurality of flow cell chemical pads 22 are positioned in a pattern across the substantially flat surface 38.

The substantially flat surface 38 may be the bottom surface of a lane 40 that is defined in the single layer substrate 24A, 24B. The lane 40 may be etched into the substrate or defined, e.g., by lithography or another suitable technique.

The plurality of flow cell chemical pads 22 are positioned in a pattern across the substantially flat surface 38.

Many different patterns for the flow cell chemical pads 22 may be envisaged, including regular, repeating, and non-regular patterns. In an example, the flow cell chemical pads 22 are disposed in a hexagonal grid for close packing and improved density. Other layouts may include, for example, rectilinear (rectangular) layouts, triangular layouts, and so forth. In some examples, the layout or pattern can be an x-y format of flow cell chemical pads 22 that are in rows and columns. In some other examples, the layout or pattern can be a repeating arrangement of flow cell chemical pads 22 separated by regions of the substantially flat substrate 38. In still other examples, the layout or pattern can be a random arrangement of flow cell chemical pads 22. The pattern may include stripes, swirls, lines, triangles, rectangles, circles, arcs, checks, diagonals, arrows, and/or squares.

The layout or pattern of the flow cell chemical pads 22 may be characterized with respect to the density of the flow cell chemical pads 22 (e.g., number of flow cell chemical pads 22) in a defined area. For example, the flow cell chemical pads 22 may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density of flow cell chemical pads 22 can be between one of the lower values and one of the upper values selected from the ranges above. As examples, a high density array may be characterized as having flow cell chemical pads 22 separated by less than about 100 nm, a medium density array may be characterized as having flow cell chemical pads 22 separated by about 400 nm to about 1 μm, and a low density array may be characterized as having chemical pads 22 separated by greater than about 1 μm. While example densities have been provided, it is to be understood that any suitable densities may be used. In some instances, it may be desirable for the spacing between flow cell chemical pads 22 to be even greater than the examples listed herein.

The layout or pattern of the flow cell chemical pads 22 may also or alternatively be characterized in terms of the average pitch, or the spacing from the center of one flow cell chemical pad 22 to the center of an adjacent flow cell chemical pad 22 (center-to-center spacing) or from the left edge of one flow cell chemical pad 22 to the right edge of an adjacent flow cell chemical pad 22 (edge-to-edge spacing). The pattern can be regular, such that the coefficient of variation around the average pitch is small, or the pattern can be non-regular in which case the coefficient of variation can be relatively large. In either case, the average pitch can be, for example, about 50 nm, about 0.1 μm, about 0.5 μm, about 1 μm, about 5 μm, about 10 μm, about 100 μm, or more or less. The average pitch for a particular pattern of flow cell chemical pads 22 can be between one of the lower values and one of the upper values selected from the ranges above. In an example, the flow cell chemical pads 22 have a pitch (center-to-center spacing) of about 1.5 μm. While example average pitch values have been provided, it is to be understood that other average pitch values may be used.

The flow cell chemical pads 22 may have any suitable shape, geometry and dimensions, which may depend, at least in part, on the pre-clustered particles 10 that is to be captured by the flow cell chemical pads 22.

The flow cell chemical pads 22 may include a chemical capture agent or an electrostatic capture agent to attach to the surface attachment mechanism of the pre-clustered particles 10′, 11′.

The flow cell chemical pads 22 may include any example of the chemical capture agent set forth herein that can be deposited on or otherwise attached to predefined locations of the substantially flat surface 38. In one example, the chemical capture agent may be deposited, e.g., using microcontact printing, aerosol printing, etc., in a desirable location on the substantially flat surface 38 to form the chemical pads 22. In another example, a sacrificial pad (e.g. mask, photoresist, etc.) may be used to define the space/location where the chemical capture agent will be deposited. The chemical capture agent may then be deposited, and the sacrificial pad removed (e.g., via lift-off, dissolution, or another suitable technique). In this example, the chemical capture agent may form a monolayer or thin layer of the chemical capture agent. In still another example, a polymer grafted with primers complementary to the capture primer(s) on the pre-clustered particles 10′, 11′ may be selectively applied to the substantially flat surface 38 to form the chemical captures sites.

In other examples, the flow cell chemical pads 22 may include any example of the electrostatic capture agent that includes a counter ion of the charged polymer of the pre-clustered particles 10′, 11′. An example of the electrostatic capture agents set forth herein that can be deposited on predefined locations of the substantially flat surface 38. For example, electrode materials may be deposited using chemical vapor deposition, masking and deposition, or another suitable technique to form the chemical pads 22. When electrostatic capture agents are used to form the chemical pads 22, the substrate 24A, 24B may include additional circuitry to address the individual chemical pads 22.

In the example of FIG. 3, areas of the substantially flat surface 38 that do not contain the chemical pads 22 function as interstitial regions between the chemical pads 22. The interstitial regions provide space so that optical signals from respective pre-clustered particles 10′, 11′ can be resolved during sequencing.

While the example architecture shown in FIG. 3 depicts the pre-clustered particles 10′, 11′ anchored at the chemical pads 22, it is to be understood that the flow cell 20 does not include the pre-clustered particles 10′, 11′ until they are introduced thereto, e.g., during sequencing.

It will be understood that different methods may be used to generate the flow cell architecture shown in FIG. 2B. The various methods will now be described.

### Methods for Making Flow Cell Architecture of FIG. 28

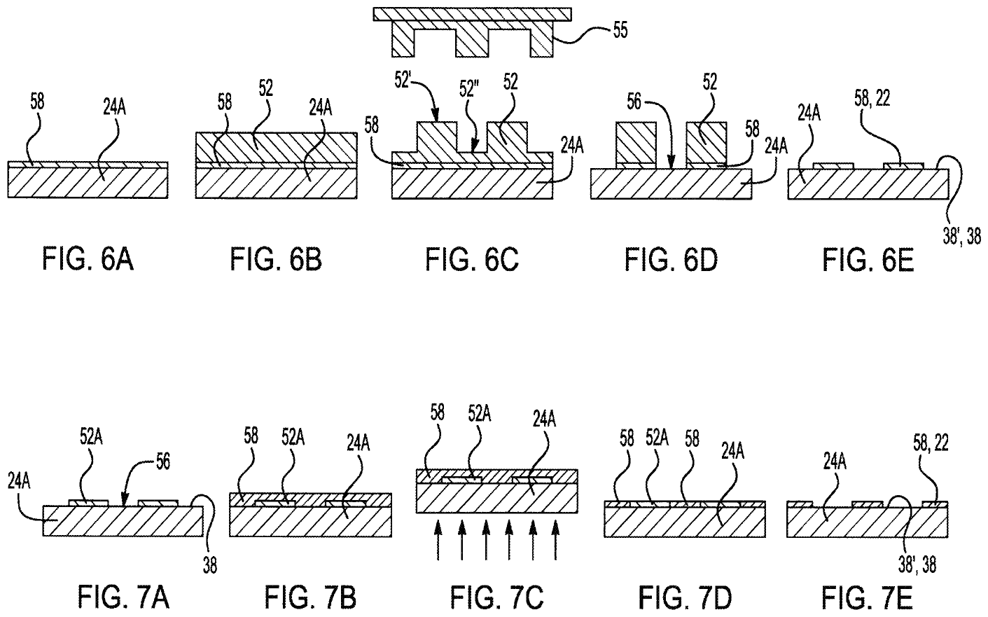

The architecture shown in FIG. 2B may be generated by a variety of methods. Several methods are shown in reference to the FIG. 4 series, the FIG. 5 series, the FIG. 6 series, and the FIG. 7 series.

The method in the FIG. 4 series includes FIG. 4A through FIG. 4E. FIG. 4A and FIG. 4B generally includes patterning a resin layer 54 to form a convex region 54′ and a concave region 54″, wherein the resin layer 54 is part of a multi-layer stack including a sacrificial layer 52 over a substrate 24A, 24B. The method may include selectively etching the resin layer 54 at the concave regions 54″ using a mixture of a CF₄ and O₂ gas at a first flow rate, thereby exposing underlying portions of the sacrificial layer 52 (not shown). The method may further include selectively etching the exposed portions of the sacrificial layer 52 using O₂ gas at a second flow rate that is lower than the first flow rate, thereby exposing portions of the substrate 24A, 24B (as shown in FIG. 4C). The method may then include applying a chemical or electrostatic capture agent 58 on the convex regions 54′ of the resin layer 54 and the exposed portions 56 of the substrate 24A, 24B (FIG. 4D); and lifting off the sacrificial layer 52, thereby leaving pads 22 of the chemical or electrostatic capture agent 58 on the substrate 24A, 24B (FIG. 4E).

The method and the various steps will now be described in more detail in reference to the respective figures.

The method begins with a multi-layer stack of materials, which includes a resin layer 54 positioned over a sacrificial layer 52 positioned over the substrate 24A, 24B (FIG. 4A and FIG. 4B). Examples of suitable substrates 24A, 24B may be any of the example substrates set forth herein. Any of the substrates 24A, 24B may be considered transparent substrates as they can transmit the wavelengths of visible light used in a sequencing operation. In some of the methods disclosed herein, it is desirable for the substrate 24A, 24B to also be transparent to the ultraviolet light used to pattern one or more materials (e.g., a photoresist) during the method.

To generate the multi-layer stack, the sacrificial layer 52 is deposited over the substrate 24A, 24B, as shown in FIG. 4A. Examples of suitable materials for the sacrificial layer 52 include lift-off resists, such as a negative or positive photoresist or poly(methyl methacrylate). An example of a suitable negative photoresist includes the NR® series photoresist (available from Futurrex). Other suitable negative photoresists include the SU-8 Series and the KMPR® Series (both of which are available from Kayaku Advanced Materials, Inc.), or the UVN™ Series (available from DuPont). Examples of suitable positive photoresists include the MICROPOSIT® S1800 series or the AZ® 1500 series, both of which are available from Kayaku Advanced Materials, Inc. Another example of a suitable positive photoresist is SPR™-220 (from DuPont). When a photoresist is used as the sacrificial layer 52, it may be developed in accordance with the type of photoresist that it is. In one example, the negative photoresist is also a lift-off resist. Examples of suitable lift-off resists include those that are commercially available from Kayaku Advanced Materials, Inc. (formerly MicroChem), which are based on a polymethylglutarimide platform. The lift-off resist may be spun on or otherwise deposited, cured, and subsequently removed at a desirable time in the process with a suitable remover. While an example has been provided, it is to be understood that any of these materials may be deposited using any suitable technique.

The resin layer 54 is then formed over the sacrificial layer 52, as shown in FIG. 4B. It is to be understood that any material that can be selectively deposited, or deposited and patterned may be used for the resin layer 54.

In one example, an inorganic oxide may be selectively applied to the substrate 24A, 24B via vapor deposition, aerosol printing, or inkjet printing. The selectively deposited inorganic oxide forms the resin layer 54, which is patterned with the concave regions 54″. Examples of suitable inorganic oxides include tantalum oxide (e.g., Ta₂O₅), aluminum oxide (e.g., Al₂O₃), silicon oxide (e.g., SiO₂), hafnium oxide (e.g., HfO₂), etc.

In another example, a polymeric resin may be applied to the substrate 24A, 24B and then patterned to form the resin layer 54. Some examples of suitable resins include a polyhedral oligomeric silsesquioxane resin, a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof. Suitable deposition techniques for the polymeric resin include chemical vapor deposition, dip coating, dunk coating, spin coating, spray coating, puddle dispensing, ultrasonic spray coating, doctor blade coating, aerosol printing, screen printing, microcontact printing, etc. Suitable patterning techniques include photolithography, nanoimprint lithography (NIL), stamping techniques, embossing techniques, molding techniques, microetching techniques, etc. For some deposition techniques, the polymeric resin may be mixed in a liquid carrier, such as propylene glycol monomethyl ether acetate (PGMEA), toluene, dimethyl sulfoxide (DMSO), tetrahydrofuran (THF), etc.

As shown in FIG. 4B, the polymeric resin is patterned to form the resin layer 54, which is patterned with a convex region 54′ and a concave region 54″. In one example, a working stamp 55 is pressed into the deposited polymeric resin while it is soft, which creates an imprint of the working stamp features in the polymeric resin. The polymeric resin may then be cured with the working stamp in place to form the patterned resin layer 54.

Curing may be accomplished by exposure to actinic radiation, such as visible light radiation or ultraviolet (UV) radiation, when a radiation-curable resin material is used; or by exposure to heat when a thermal-curable resin material is used. Curing may promote polymerization and/or cross-linking. As an example, curing may include multiple stages, including a softbake (e.g., to drive off any liquid carrier that may be used to deposit the resin) and a hardbake. The softbake may take place at a lower temperature, ranging from about 50° C. to about 150° C. The duration of the hardbake may last from about 5 seconds to about 10 minutes at a temperature ranging from about 100° C. to about 300° C. Examples of devices that can be used for softbaking and/or hardbaking include a hot plate, oven, etc.

After curing, the working stamp 55 is released. This creates topographic features in the resin layer 54. In this example method, the working stamp 55 does not extend through the entire depth of the polymeric resin, and thus the underlying sacrificial layer 52 is not exposed after imprinting (as shown in FIG. 4B).

The method proceeds with FIG. 4C. In this example, the multi-layer stack is then selectively etched in the concave region 54″ using a mixture of CF₄ and O₂ gas at a certain ratio and flow rate, thereby exposing portions of the sacrificial layer 52. In this example, etching exposes portions of the sacrificial layer 52 positioned over substrate 24A, 24B at the concave regions 54″, and the substrate 24A, 24B remains unetched. This effectively extends the convex region 54′ down to the sacrificial layer 52 positioned over substrate 24A, 24B.

In this example, after the resin layer 54 is etched at the concave regions 54″, exposed portion(s) of the sacrificial layer 52 (at the concave regions 54″) is/are selectively etched using O₂ gas at a second flow rate that is lower than the first flow rate, thereby exposing portions of the substrate 24A, 24B (at the concave regions 54″).

As shown at FIG. 4D, the chemical or electrostatic capture agent 58 is then applied over the remaining multi-layer stack. More particularly, the chemical or electrostatic capture agent 58 is applied on the exposed portions 56 of the substrate 24A, 24B (at the concave regions 54″) and the convex regions 54′ of the resin layer 54. The chemical or electrostatic capture agent 58 may include any example of a chemical or electrostatic capture agent set forth herein that can be deposited on the surface of the substrate 24A, 24B. The attachment (e.g., covalent, non-covalent) between the chemical or electrostatic capture agent 58 and the underlying substrate 24A, 24B will depend upon the chemistry of agent 58 and substrate 24A, 24B. For example, PAZAM having pre-grafted primers, one of which is complementary to the particle's capture primer, may be covalently attached to a silanized substrate.

Lift-off of the remaining sacrificial layer 52 may then be performed. As shown in FIG. 4E, the lift-off process removes i) at least 99% of the sacrificial layer 52 and ii) the patterned resin layer 54 (i.e., convex regions 54′) and the chemical or electrostatic capture agent 58 that overlies or is attached to the patterned resin layer 54. The lift-off process may involve an organic solvent that is capable of dissolving or otherwise lifting off the sacrificial layer 52. As examples, an aluminium sacrificial layer 52 can be removed in acidic (e.g., nitric acid based) or basic (e.g., KOH based) conditions, a copper sacrificial layer 52 can be removed using FeCl₃, and a copper, gold or silver sacrificial layer 52 can be removed in an iodine and iodide solution. Lift-off resists and/or photoresists may be removed using a suitable remover/resist stripper for the particular type of resist that is used.

Lift-off of the remaining sacrificial layer 52 and any overlying layers leaves pads 22 of the chemical or electrostatic capture agent 58 at spatially separated locations relative to one another on the surface 38 of the substrate 24A, 24B. The pads 22 of the chemical or electrostatic capture agent 58 remain intact over the substrate 24A, 24B, in part because the chemical or electrostatic capture agent 58 is attached (e.g., covalently) to the substrate 24A, 24B. It is to be understood that any suitable type of bonding may be used to attach the pads 22 to the surface 38 of the substrate 24A, as long as the bond is strong and stable so that the pad 22 can survive the sequencing conditions.

While only two pads 22 of the chemical or electrostatic capture agent 58 are shown in FIG. 4E, it is to be understood that the method described in reference to FIG. 4A through FIG. 4E may be performed to generate an array of pads 22 of the chemical or electrostatic capture agent 58, separated by interstitial regions 38′ across the substantially flat surface 38 of the substrate 24A, 24B.

In one specific example, the method comprises generating a plurality of chemical pads 22 (as described above) that are spatially separated from one another on a substantially flat surface 38 of a substrate 24A, 24B, wherein each of the chemical pads 22 includes a chemical capture agent.

FIG. 5A through 5G illustrates two different examples of the method for making the flow cell architecture of FIG. 2B, which includes the pads 22 of the chemical or electrostatic capture agent 58. One example method is shown in FIG. 5A through FIG. 5E, and another example method is shown in FIG. 5A through FIG. 5C, FIG. 5F, and FIG. 5G.

The example shown in FIG. 5A through FIG. 5E generally includes patterning a polymeric resin to form a resin layer 54 including a convex region 54′ and a concave region 54″, wherein the resin layer 54 is part of a multi-layer stack including a sacrificial layer 52 over a chemical or electrostatic capture agent 58 over a substrate 24A, 24B (FIG. 5A, FIG. 5B, and FIG. 5C). The method may include selectively etching at the concave regions 54″ using a mixture of CF₄ and O₂ gas at a certain ratio (10/1) with a relatively low chamber pressure (˜6 mTorr) and a relatively high radio frequency power (˜210 W), thereby exposing underlying portions of the sacrificial layer 52 (not shown). The method may further include selectively etching the exposed portions of the sacrificial layer 52 and underlying portions of the chemical or electrostatic capture agent 58 using the O₂ gas at a second flow rate that is lower than the first flow rate, thereby exposing portions of the substrate 24A, 24B (as shown in FIG. 5D). The method may then include lifting off the sacrificial layer 52, thereby leaving pads 22 of the chemical or electrostatic capture agent 58 on the substrate 24A, 24B (FIG. 5E).

The method and the various steps will now be described in more detail in reference to the respective figures.

The method begins with a multi-layer stack of materials, which includes a resin layer 54 positioned over a sacrificial layer 52 positioned over a chemical or electrostatic capture agent 58 positioned over the substrate 24A, 24B.

To generate the multi-layer stack, the chemical or electrostatic capture agent 58 is deposited over the substrate 24A, 24B, as shown in FIG. 5A. The sacrificial layer 52 is then deposited over the chemical or electrostatic capture agent 58 (FIG. 5B). Examples of suitable materials for the sacrificial layer 52, chemical or electrostatic capture agent 58, and substrate 24A, 24B can be selected from any materials disclosed herein. These materials for the sacrificial layer 52 and the chemical or electrostatic capture agent 58 may be deposited using any suitable technique disclosed herein.

The resin layer 54 is then formed over the sacrificial layer 52, as shown in FIG. 5C. It is to be understood that any material that can be selectively deposited, or deposited and patterned may be used for the resin layer 54. Examples of suitable materials for the resin layer 54 can be selected from any material disclosed herein (e.g., inorganic oxides, polymeric resins).

In the example shown in FIG. 5C, a polymeric resin is deposited and patterned to form the resin layer 54 having a convex region 54′ and a concave region 54″. In one example, a working stamp 55 is pressed into the polymeric resin while it is soft, which creates an imprint of the working stamp features in the polymeric resin. The polymeric resin may then be cured with the working stamp in place. Curing may be accomplished as described herein in reference to FIG. 4B.

After curing, the working stamp 55 is released. This creates topographic features in the resin layer 54. In this example method, the working stamp 55 does not extend through the entire depth of the polymeric resin, and thus the underlying sacrificial layer 52 is not exposed after imprinting (as shown in FIG. 5C).

One example method proceeds with FIG. 5D. In this example, the multi-layer stack is then selectively etched in the concave region 54″ using a mixture of CF₄ and O₂ gas at a certain ratio (10/1) with a relatively low chamber pressure (˜6 mTorr) and a relatively high radio frequency (RF) power (˜210 W), thereby exposing portions of the sacrificial layer 52. In this example, etching exposes portions of the sacrificial layer 52 positioned over substrate 24A, 24B at the concave regions 54″, and the substrate 24A, 24B remains unetched. This effectively extends the convex region 54′ down to the sacrificial layer 52 positioned over substrate 24A, 24B.

In this example, after the resin layer 54 is etched at the concave regions 54″, portion(s) of the sacrificial layer 52 and chemical or electrostatic capture agent 58 (that had been underlying the concave regions 54″) is/are selectively etched using the O₂ gas at a second flow rate that is lower than the flow rate (used for the mixture of CF₄ and O₂ gas), thereby exposing portions of the substrate 24A, 24B (that had been underlying the exposed portions of the sacrificial layer 52).

Lift-off of the remaining sacrificial layer 52 may then be performed. As shown in FIG. 5E, the lift-off process removes i) at least 99% of the sacrificial layer 52 and ii) the patterned resin layer 54 (i.e., convex regions 54′). The lift-off process may involve an organic solvent that is capable of dissolving or otherwise lifting off the sacrificial layer 52. For example, a lift-off resist sacrificial layer 52 and/or a photoresist sacrificial layer 52 may be removed using a suitable remover/resist stripper for the particular type of resist that is used.

Lift-off of the remaining sacrificial layer 52 and the overlying resin layer 54 leaves pads 22 of the chemical or electrostatic capture agent 58 at spatially separated locations relative to one another on the surface 38 of the substrate 24A, 24B. The pads 22 of the chemical or electrostatic capture agent 58 remain intact over the substrate 24A, 24B, in part because the chemical or electrostatic capture agent 58 is strongly attached, via covalent bonding or non-covalent adhesion, to the substrate 24A, 24B.

While only two pads 22 of the chemical or electrostatic capture agent 58 are shown in FIG. 5E, it is to be understood that the method described in reference to FIG. 5A through FIG. 5E may be performed to generate an array of pads 22 of the chemical or electrostatic capture agent 58, separated by interstitial regions 38′ across the substantially flat surface 38 of the substrate 24A, 24B.

Another example method is shown at FIG. 5A through FIG. 5C and FIG. 5F through FIG. 5G. The processes of FIG. 5A through FIG. 5C may be performed as described herein.

Referring back to FIG. 5C, another example of the method proceeds from FIG. 5C to FIG. 5F and then to FIG. 5G.

In FIG. 5F, removing the resin layer 54 at the concave regions 54″, the sacrificial layer 52 (at the concave regions 54″), and the chemical or electrostatic capture agent 58 (at the concave regions 54″) involves reactive ion etching. It is to be understood that reactive ion etching involves: anisotropically etching the resin layer 54 (at the concave regions 54″), the sacrificial layer 52 (at the concave regions 54″), and the chemical or electrostatic capture agent 58 (at the concave regions 54″); and generating an undercut profile by isotropically etching some of the sacrificial layer 52 and the chemical or electrostatic capture agent 58 that underlie the resin layer 54 at the convex regions 54′. In one example, the isotropic etching to generate the undercut profile can be achieved by a third O₂ gas flow at a higher chamber pressure (˜50 mTorr) and a lower RF power (˜100 W). This method allows tunable etching conditions resulting in smaller sized chemical pads 22 and greater interstitial regions 38′ between the chemical pads 22, which may lead to a size-matched loading for smaller particles 10′, 11′ and/or improved signal resolution during sequencing.

Lift-off of the remaining sacrificial layer 52 and the overlying resin layer 54 may then be performed. As shown in FIG. 5G, the lift-off process removes i) at least 99% of the sacrificial layer 52 and ii) the patterned resin layer 54 overlying the sacrificial layer 52. The lift-off process may involve an organic solvent that is capable of dissolving or otherwise lifting off the sacrificial layer 52. For example, a lift-off resist sacrificial layer 52 and/or a photoresist sacrificial layer 52 may be removed using a suitable remover/resist stripper for the particular type of resist that is used.

Lift-off of the remaining sacrificial layer 52 and the overlying resin layer 54 leaves pads 22 of the chemical or electrostatic capture agent 58 at spatially separated locations relative to one another on the surface of the substrate 24A, 24B. The pads 22 of the chemical or electrostatic capture agent 58 remain intact over the substrate 24A, 24B, in part because the chemical or electrostatic capture agent 58 is strongly attached, via covalent bonding or non-covalent adhesion, to the substrate 24A, 24B.

While only two pads 22 of the chemical or electrostatic capture agent 58 are shown in FIG. 5G, it is to be understood that the method described in reference to FIG. 5A through FIG. 5C and FIG. 5F through FIG. 5G may be performed to generate an array of pads 22 of the chemical or electrostatic capture agent 58, separated by interstitial regions 38′ across the surface 38 of the substrate 24A, 24B.

FIG. 6A through FIG. 6E illustrate another example of the method for making the flow cell architecture of FIG. 2B, which includes the pads 22 of the chemical or electrostatic capture agent 58. The example shown in FIG. 6A through FIG. 6E generally includes patterning a sacrificial layer 52 to form a convex region 52′ and a concave region 52″, wherein the sacrificial layer 52 is part of a multi-layer stack including a chemical or electrostatic capture agent 58 over a substrate 24A, 24B (FIG. 6A through FIG. 6C). In this example, the sacrificial layer 52 effectively serves as the patternable resin layer 54 of the other examples. As such, this example method utilizes fewer materials, which may lead to a simpler, cleaner lift-off process. The method may include selectively etching the sacrificial layer 52 at the concave regions 52″ and underlying portions of the chemical or electrostatic capture agent 58 using an O₂ gas at a predetermined flow rate, pressure and RF power, thereby exposing portions of the substrate 24A, 24B. The method may then include lifting off the sacrificial layer 52, thereby leaving pads 22 of the chemical or electrostatic capture agent 58 on the substrate 24A, 24B (FIG. 6E).

The method and the various steps will now be described in more detail in reference to the respective figures.

The method begins with a multi-layer stack of materials, which includes a sacrificial layer 52 positioned over a chemical or electrostatic capture agent 58 positioned over the substrate 24A, 24B (FIG. 6B).

To generate the multi-layer stack, the chemical capture agent 58 is deposited over the substrate 24A, 24B, as shown in FIG. 6A. The sacrificial layer 52 is then deposited over the chemical or electrostatic capture agent 58 (FIG. 6B). Examples of suitable materials for the sacrificial layer 52, chemical or electrostatic capture agent 58, and substrate 24A, 24B can be selected from any materials disclosed herein. The materials for the chemical or electrostatic capture agent 58 and the sacrificial layer 52 may be deposited using any suitable technique disclosed herein.

As shown in FIG. 6C, the sacrificial layer 52 is patterned to form a convex region 52′ and a concave region 52″. Thus, in this example, the sacrificial layer 52 may be any material that can be patterned using nanoimprint lithography, such as polydimethylglutarimide polymers or other imprintable polymer based resists. In one example, a working stamp 55 is pressed into the sacrificial layer 52 while it is soft, which creates an imprint of the working stamp features in the sacrificial layer 52. The sacrificial layer 52 may then be cured or hardened with the working stamp in place. Curing may be accomplished as described herein in reference to FIG. 4B.

After curing or hardening, the working stamp 55 is released. This creates topographic features in the sacrificial layer 52. In this example method, the working stamp 55 does not extend through the entire depth of the sacrificial layer 52, and thus the underlying chemical or electrostatic capture agent 58 is not exposed after imprinting (as shown in FIG. 6C).

The method proceeds with FIG. 6D. In this example, the multi-layer stack is then selectively etched at the concave region 52″ using an O₂ gas at a predetermined flow rate, pressure, and RF power, thereby exposing portions 56 of the substrate 24A, 24B at the concave region 52″. This etching step removes some of the sacrificial layer 52 and some of the chemical or electrostatic capture agent 58.

Lift-off of the remaining sacrificial layer 52 may then be performed. As shown in FIG. 6E, the lift-off process removes at least 99% of the sacrificial layer 52. The lift-off process may involve an organic solvent that is capable of dissolving or otherwise lifting off the sacrificial layer 52. For example, a lift-off resist sacrificial layer 52 and/or a photoresist sacrificial layer 52 may be removed using a suitable remover/resist stripper for the particular type of resist that is used.

Lift-off of the remaining sacrificial layer 52 leaves pads 22 of the chemical or electrostatic capture agent 58 58 at spatially separated locations relative to one another on the surface 38 of the substrate 24A, 24B. The pads 22 of the chemical capture agent 58 remain intact over the substrate 24A, 24B, in part because the chemical or electrostatic capture agent 58 is strongly attached, via covalent bonding or non-covalent adhesion, to the substrate 24A, 24B.

While only two pads 22 of the chemical or electrostatic capture agent 58 are shown in FIG. 6E, it is to be understood that the method described in reference to FIG. 6A through FIG. 6E may be performed to generate an array of pads 22 of the chemical or electrostatic capture agent 58, separated by interstitial regions 38′ across the surface 38 of the substrate 24A, 24B.

FIG. 7A through FIG. 7E illustrate yet another example of the method for making the flow cell architecture of FIG. 2B, which includes the pads 22 of the chemical or electrostatic capture agent 58. The example shown in FIG. 7A through FIG. 7E begins with generating a plurality of sacrificial pads 52A on the substantially flat surface of a substrate 24A, 24B such that regions or exposed portions 56 of the substrate 24A, 24B separate each of the plurality of sacrificial pads 52A (FIG. 7A). The method may then include applying a chemical or electrostatic capture agent 58 on the sacrificial pads 52A and the exposed portions 56 of the substrate 24A, 24B (FIG. 7B). The method may include introducing ultraviolet light through the substrate 24A, 24B, whereby portions of the chemical or electrostatic capture agent 58 overlying the exposed regions 56 are cured and other portions of the chemical or electrostatic capture agent 58 overlying the plurality of sacrificial pads 52A are uncured (FIG. 7C). Thus, in this example method, the chemical or electrostatic capture agent 58 is photocurable. The method may then include removing the uncured portions of the chemical or electrostatic capture agent 58 (FIG. 7D) and removing the plurality of sacrificial pads 52A (FIG. 7E).

The method and the various steps will now be described in more detail in reference to the respective figures.

The method begins with a multi-layer stack of materials, which includes a chemical or electrostatic capture agent 58 positioned over a plurality of sacrificial pads 52A positioned over a substrate 24A, 24B.

In one example, to generate this multi-layer stack, the sacrificial pads 52A are formed over the substrate 24A, 24B, as shown in FIG. 7A. Any example of the sacrificial materials described herein may be used for the sacrificial pads 52A in this example method, as long as it is opaque or non-transparent to the light energy being used for backside exposure. In an example, the sacrificial pads 52A may alternatively comprise any ultraviolet opaque or non-transparent metal or ultraviolet opaque semi-metal, such as titanium, chromium, platinum, aluminum, copper, silicon, etc. In one example, the sacrificial pads 52A comprise chromium.

A selective deposition technique, such as chemical vapor deposition (CVD) and variations thereof (e.g., low-pressure CVD or LPCVD), atomic layer deposition (ALD), and masking techniques, may be used to deposit the sacrificial material is desirable positions to form the sacrificial pads 52A so that regions or exposed portions 56 of the substantially flat surface 38 of the substrate 24A, 24B separate each of the plurality of sacrificial pads 52A.

As shown in FIG. 7B, the chemical or electrostatic capture agent 58 is then applied over the sacrificial pads 52A and over the exposed portions 56 of substrate 24A, 24B surface using any suitable deposition technique. In this example, any chemical or electrostatic capture agent 58 that can be cured by UV light exposure may be used. One example of the electrostatic capture agent 58 for this example method is a negative photoresist with electrocharges (e.g., AZ® 125 nXT resist developed by MicroChemicals Inc.)

Referring to FIG. 7C, this example of the method further includes directing UV light through the substrate 24A, 24B, whereby the sacrificial pads 52A block at least 75% of the UV light that is transmitted through the substrate 24A, 24B, so that the chemical or electrostatic capture agent 58 overlying the sacrificial pads 52A remain uncured. Additionally, the substrate 24A, 24B transmits at least 25% of the UV light to portions of the chemical or electrostatic capture agent 58 that are positioned over the exposed portions 56 of the substrate 24A, 24B. The UV light cures the portions of the chemical or electrostatic capture agent 58 exposed thereto.

The uncured portions of the chemical or electrostatic capture agent 58 are removed using a suitable developer (FIG. 7D), followed by removal of the sacrificial pads 52A (FIG. 7E). Removal of the sacrificial pads 52A may be performed with an organic solvent or remover/resist stripper that is capable of dissolving or otherwise lifting off the sacrificial pads 52A. As shown in FIG. 7E, the lift-off process removes at least 99% of the sacrificial pads 52A.

Sacrificial pad 52A removal leaves pads 22 of the chemical or electrostatic capture agent 58 at spatially separated locations relative to one another on the surface of the substrate 24A, 24B. The pads 22 of the chemical or electrostatic capture agent 58 remain intact over the substrate 24A, 24B, in part because the chemical or electrostatic capture agent 58 are covalently attached to the substrate 24A, 24B.

While only two pads 22 of the chemical or electrostatic capture agent 58 are shown in FIG. 7E, it is to be understood that the method described in reference to FIG. 7A through FIG. 7E may be performed to generate an array of pads 22 of the chemical or electrostatic capture agent 58 separated by interstitial regions 38′ across the surface 38 of the substrate 24A, 24B.

While not shown, the methods described in the FIG. 4 series, the FIG. 5 series, the FIG. 6 series, and the FIG. 7 series may include PAZAM as the chemical or electrostatic capture agent 58 such that it can attach, e.g., via click chemistry, to the flow cell attachment mechanism (specifically an alkene or alkyne functional group of the hydrogel 14 or hydrogel core 12′) of the pre-clustered particles 10′, 11′.

In other examples, the method further comprises exposing the plurality of PAZAM chemical pads 22 to an azide reducing agent to convert at least some azide groups of the poly(N-(5-azidoacetamidylpentyl)acrylamide-co -acrylamide) to amine groups. The azide reducing agent may be selected from the group consisting of a phosphine and a phosphite. In one example, the azide reducing agent may be the phosphine selected from the group consisting of Tris(2-carboxyethyl)phosphine hydrochloride) (TCEP) and Tris(hydroxypropyl)phosphine; and exposing the plurality of chemical pads 22 to the azide reducing agent takes place at a temperature ranging from about 50° C. to about 60° C. for a time ranging from about 5 minutes to about 10 minutes. In one example, the method may begin by exposing the flow cell surface (having the PAZAM chemical pads 22 formed thereon using any of the example methods disclosed herein) to the azide reducing agent. In this example, the azide reducing agent is allowed to incubate on the flow cell surface to reduce at least some of the azide functional groups of the chemical pads 22 to amine functional groups. As noted above, in an example, the azide reducing agent is phosphine (e.g., Tris(2-carboxyethyl)phosphine hydrochloride) (TCEP), Tris(hydroxypropyl)phosphine, or another suitable phosphine) or phosphite, and the reduction reaction occurs at a temperature ranging from about 50° C. to about 60° C. for a time ranging from about 5 minutes to about 10 minutes. The reduction reaction reduces the azide functional groups to amine functional groups, which can react with the 3′ end groups of the primers 16A, 16B on the pre-clustered particle surface.

### Kits Including the Functionalized Particles

Any example of the flow cell 20 and the functionalized particles 10, 11 may be part of a sequencing kit. An example of the kit includes the flow cell 20, which includes a plurality of chemical pads 22, and a suspension, which includes a liquid carrier and a plurality of the functionalized particles 10, 11 dispersed throughout the liquid carrier.

Any example of the flow cell 20 disclosed herein may be used, as long as the chemical pads 22 are selected to be able to attach the surface attachment mechanism of the functionalized particles 10, 11 in the kit.

Any example of the functionalized particles 10, 11 disclosed herein may be used in the suspension. Examples of the liquid carrier include a buffer (e.g., a Tris-HCl buffer or 0.5× saline sodium citrate (SSC) buffer), acetic acid, acetone, acetonitrile, benzene, butanol, diethylene glycol, diethyl ether, dimethyl formamide, ethanol, glycerin, methane, pyridine, triethyl amine, etc. Surfactants/dispersants, such as sodium dodecyl sulfate (SDS), (CTAB) may also be included. This suspension may be used for off-flow cell template strand preparation and amplification, and then may be incorporated into the flow cell for sequencing. In the kit, the mechanism of the functionalized particles 10, 11 is selected to be able to anchor the pre-clustered particles 10′, 11′ to the chemical pads 22 of the flow cell 20 in the kit.

In one example, the sequencing kit may further comprise an azide reducing agent to convert at least some azide groups of the poly(N-(5-azidoacetamidylpentyl)acrylamide-co-acrylamide) to amine groups.

### Sequencing Method

The pre-clustered particles 10′, 11′ may be used in sequencing on the flow cell 20. After amplification and cluster generation, the suspension including the pre-clustered particles 10′, 11′ (which includes a cluster of the template strands 18), may be introduced into the flow cell 20 including the plurality of chemical pads 22, whereby at least some of the pre-clustered particles 10′, 11′ respectively attach to at least some of the chemical pads 22. As described herein, each of the pre-clustered particles 10′, 11′ includes the flow cell surface attachment mechanism that specifically binds, attaches, or is otherwise attracted (e.g., electrostatically, etc.) to the chemical pads 22 on the flow cell surface 38. The suspension may be allowed to incubate for a predetermined time in the flow cell 20 to allow the pre-clustered particles 10′, 11′ to become anchored.

When electrostatic chemical pads are used, the individual chemical pads 22 may be electrically addressed to attract the oppositely charged pre-clustered particles 10′, 11′ toward the individual chemical pads 22.

After incubation, a wash cycle may be performed to remove any unanchored pre-clustered particles 10′, 11′ and the liquid carrier of the suspension.

Sequencing primers may then be introduced to the flow cell 20. The sequencing primers hybridize to a complementary portion of the sequence of the template strands 18 that are attached to the pre-clustered particles 10′, 11′ (which are now anchored to the chemical pads 22 on the flow cell surface 38). These sequencing primers render the template strands 18 ready for sequencing.

An incorporation mix including labeled nucleotides may then be introduced into the flow cell 20, e.g., via an input port. In addition to the labeled nucleotides, the incorporation mix may include water, a buffer, and polymerases capable of nucleotide incorporation. When the incorporation mix is introduced into the flow cell 20, the mix enters the flow channel 26, and contacts the anchored and sequence ready pre-clustered particles 10′, 11′.

The incorporation mix is allowed to incubate in the flow cell 20, and labeled nucleotides (including optical labels) are incorporated by respective polymerases into the nascent strands along the template strands 18 on each of the pre-clustered particles 10′, 11′. During incorporation, one of the labeled nucleotides is incorporated, by a respective polymerase, into one nascent strand that extends one sequencing primer and that is complementary to one of the template strands 18. Incorporation is performed in a template strand dependent fashion, and thus detection of the order and type of labeled nucleotides added to the nascent strand can be used to determine the sequence of the template strand 18. Incorporation occurs in at least some of the template strands 18 across the pre-clustered particles 10′, 11′ during a single sequencing cycle.

The incorporated labeled nucleotides may include a reversible termination property due to the presence of a 3′ OH blocking group, which terminates further sequencing primer extension once the labeled nucleotide has been added. After a desired time for incubation and incorporation, the incorporation mix, including non-incorporated labeled nucleotides, may be removed from the flow cell 20 during a wash cycle. The wash cycle may involve a flow-through technique, where a washing solution (e.g., buffer) is directed into, through, and then out of flow channel 26, e.g., by a pump or other suitable mechanism.

Without further incorporation taking place, the most recently incorporated labeled nucleotides can be detected through an imaging event. During the imaging event, an illumination system may provide an excitation light to the flow cell 20. The optical labels of the incorporated labeled nucleotides emit optical signals in response to the excitation light.

After imaging is performed, a cleavage mix may then be introduced into the flow cell 20. In an example, the cleavage mix is capable of i) removing the 3′ OH blocking group from the incorporated nucleotides, and ii) cleaving the optical label from the incorporated nucleotide. Examples of 3′ OH blocking groups and suitable de-blocking agents/components in the cleavage mix may include: ester moieties that can be removed by base hydrolysis; allyl-moieties that can be removed with Nal, chlorotrimethylsilane and Na₂S₂O₃ or with Hg(II) in acetone/water; azidomethyl which can be cleaved with phosphines, such as tris(2-carboxyethyl)phosphine (TCEP) or tri(hydroxypropyl)phosphine (THP); acetals, such as tert-butoxy-ethoxy which can be cleaved with acidic conditions; MOM (—CH₂OCH₃) moieties that can be cleaved with LiBF₄ and CH₃CN/H₂O; 2,4-dinitrobenzene sulfenyl which can be cleaved with nucleophiles such as thiophenol and thiosulfate; tetrahydrofuranyl ether which can be cleaved with Ag(I) or Hg(II); and 3′ phosphate which can be cleaved by phosphatase enzymes (e.g., polynucleotide kinase). Examples of suitable optical label cleaving agents/components in the cleavage mix may include: sodium periodate, which can cleave a vicinal diol; phosphines, such as tris(2-carboxyethyl)phosphine (TCEP) or tris(hydroxypropyl)phosphine (THP), which can cleave azidomethyl linkages; palladium and THP, which can cleave an allyl; bases, which can cleave ester moieties; or any other suitable cleaving agent.

Additional sequencing cycles may then be performed until the template strands 18 are sequenced.

To further illustrate the present disclosure, examples are given herein. It is to be understood that these examples are provided for illustrative purposes and are not to be construed as limiting the scope of the present disclosure.

### NON-LIMITING WORKING EXAMPLES

### Example 1

A multi-layer stack of materials similar to that shown in FIG. 5B and FIG. 5C was prepared. The stack included a glass substrate, a pre-grafted PAZAM layer (about 20 nm thick) as the layer to form the chemical pads, a lift-off resist (i.e., a polydimethylglutarimide-based polymer) as the sacrificial layer (about 170 nm thick), and a nanoimprint lithography resin (e.g., an epoxy siloxane based resin) as the resin layer.

After the resin layer was deposited, it was imprinted with a working stamp and was cured. The posts (convex regions) had a height of about 300 nm and were separated from each other at concave regions. The residual resin layer in the concave regions was about 240 nm thick.

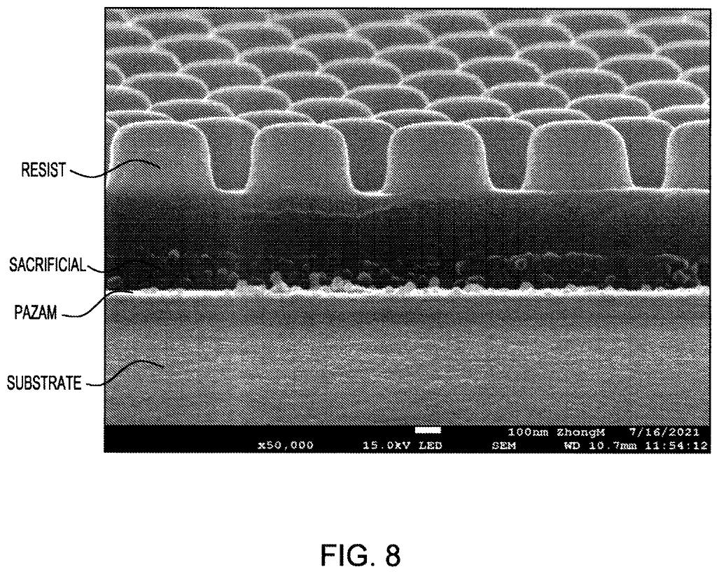

A scanning electron micrograph a portion of the multi-layer stack after the resin layer was patterned was taken from the cross-section of the sample. This is shown in FIG. 8. This image clearly shows that the various layers are intact after the resin layer is patterned.

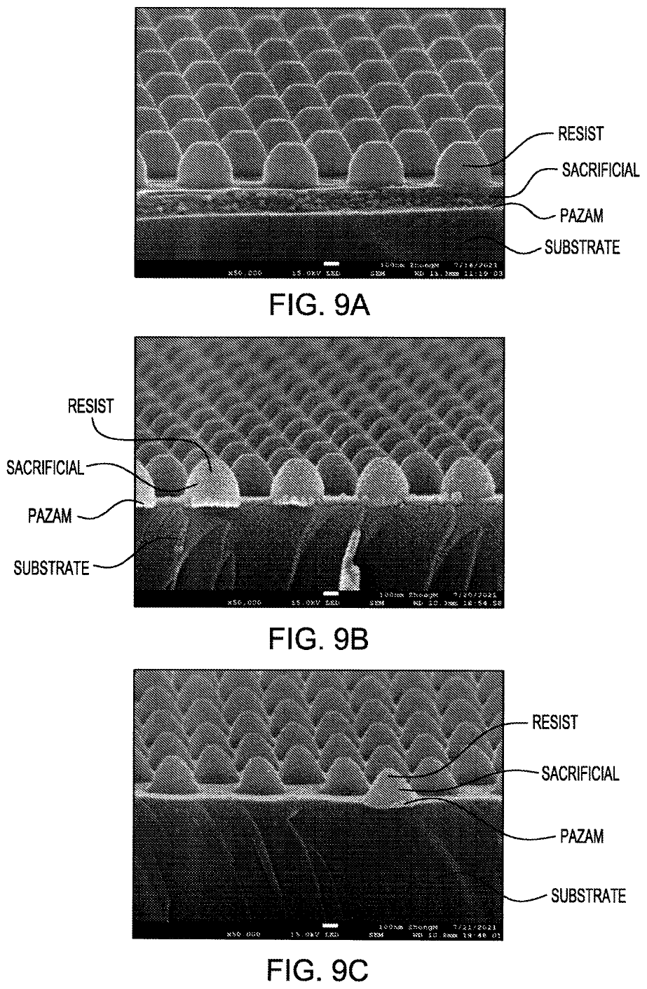

The multi-layer stack was then exposed to reactive ion etching to remove portions of the resin layer, the lift-off resist, and the pre-grafted PAZAM layer. Reactive ion etching was performed using an anisotropic etch (in the vertical direction), and the entire material stack (at both the concave regions and the convex regions) was exposed to etching. The reactive ion etching process utilized a mixture of CF₄ (flow rate of about 20 sccm) and O₂ (flow rate of about 2 sccm) with a controlled chamber pressure of 6 mTorr and a RF power of 210 W and was initially performed for 9 minutes. A scanning electron micrograph of a portion of the multi-layer stack was taken from the side after 9 minutes of etching. This is shown in FIG. 9A. As illustrated, almost the entire resin layer was removed at the concave regions and the convex regions were partially removed.

The reactive ion etching process was continued for another 3 minutes (total of 12 minutes). Another scanning electron micrograph of the portion of the multi-layer stack was taken from the side after 12 minutes of etching. This is shown in FIG. 9B. As illustrated, the resin layer was completely removed, and almost the entire sacrificial layer was removed in the concave regions. Convex regions of the sacrificial layer and PAZAM layer remained intact under the resin layer.

The reactive ion etching process was again continued for another 2 minutes (total of 14 minutes). Another scanning electron micrograph of the portion of the multi-layer stack was taken from the cross section after 14 minutes of etching. This is shown in FIG. 9C. As illustrated, the sacrificial layer and PAZAM layer were completely removed at the concave regions, thus exposing the substrate surface at the concave regions. The convex regions of sacrificial layer and PAZAM layer remained intact under the resin layer.

### Example 2

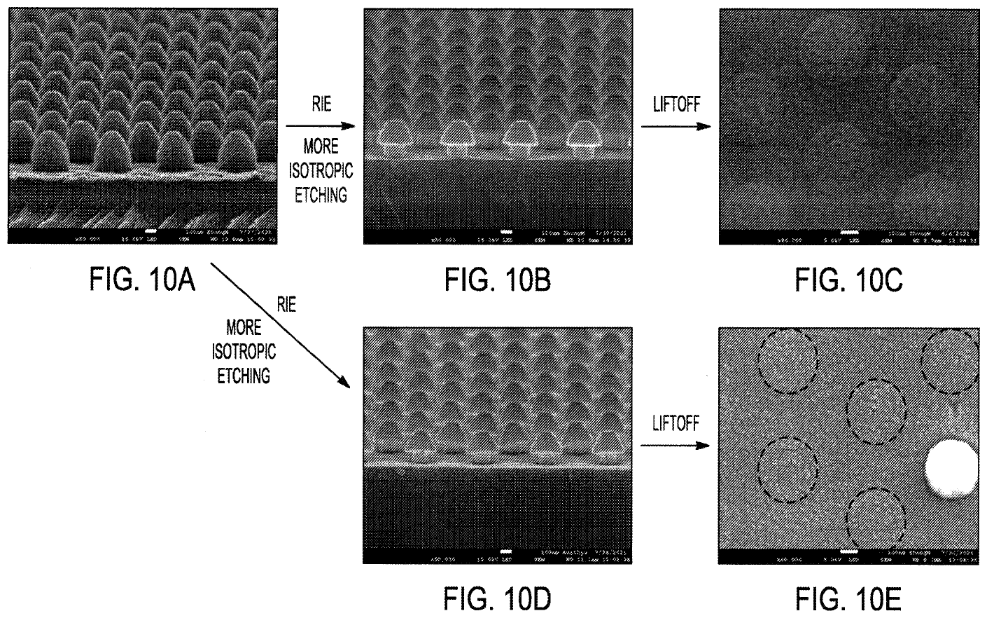

The same material stack used in Example 1 was used in this example. The resin layer was patterned as described in Example 1. The same reactive ion etching process (vertical/anisotropic) was selectively exposed to the concave regions and performed for 14 minutes to remove the resin layer, the sacrificial layer, and the pre-grafted PAZAM from the concave regions. The resulting structure is shown in FIG. 10A.

For one material stack, additional reactive ion etching was performed. The additional reactive ion etching utilized O₂ gas (flow rate of about 50 sccm, chamber pressure of about 50 mTorr and RF power of about 100 W) and was performed for 1 minute. The etching direction was altered to be universal (vertical and horizontal). This additional etching process targeted the sacrificial layer and the PAZAM beneath the resin layer, thus resulting in the undercut profile shown in FIG. 10B.

The material stack was then exposed to a lift-off reagent (Remover PG, 10 minute sonication, water rinse, N₂ dry), which removed the sacrificial layer and the remaining regions of the resin layer overlying the sacrificial layer. A top view of the remaining pre-grafted PAZAM pads is show in FIG. 10C. The pre-grafted PAZAM pads had a diameter of about 360 nm.

For another material stack, additional reactive ion etching was performed. For this material stack, the additional reactive ion etching utilized O₂ gas (flow rate of about 50 sccm, chamber pressure of about 50 mTorr and RF power of about 100 W) and was performed for 1.5 minutes. The etching direction was altered to be universal (vertical and horizontal). This additional etching process also targeted the sacrificial layer and the PAZAM beneath the resin layer, thus resulting in the undercut profile shown in FIG. 10D.

As illustrated in FIG. 10D, the resin layer at the concave regions remained intact, while the sacrificial layer and pre-grafted PAZAM layer were partially removed along the sides.

The other material stack was then exposed to the lift-off reagent (Remover PG, 10 minute sonication, water rinse, N₂ dry), which removed the sacrificial layer and the remaining regions of the resin layer overlying the sacrificial layer. A top view of the remaining pre-grafted PAZAM pads is shown in FIG. 10E (with the outline of the pads having been added for clarity). These pre-grafted PAZAM pads had a diameter of about 300 nm.

These results illustrate that etching direction and time may be used to control the size of the chemical pads.

### Example 3

The same material stack used in Example 1 was used in this example. The resin layer was patterned and etched as described in Example 1 to form PAZAM chemical pads 22. The flow cell surface with the PAZAM chemical pads was subjected to an azide reducing agent to convert at least some azide groups of the poly(N-(5-azidoacetamidylpentyl)acrylamide-co-acrylamide) to amine groups and was incubated at 60° C. for 5 minutes, followed by a water flow rinse.

Pre-clustered particles 10′ (with vinyl-PEG attached to the hydrogel coating) were suspended in a 0.5× saline sodium citrate (SSC) buffer at concentration of 0.1 mg/mL. The suspension of the pre-clustered particles 10′ was added to the flow cell channels 26 by pumping back and forth about 20 cycles at a flow rate of 60 uL/min using the cBot instrument (available from Illumina Inc).

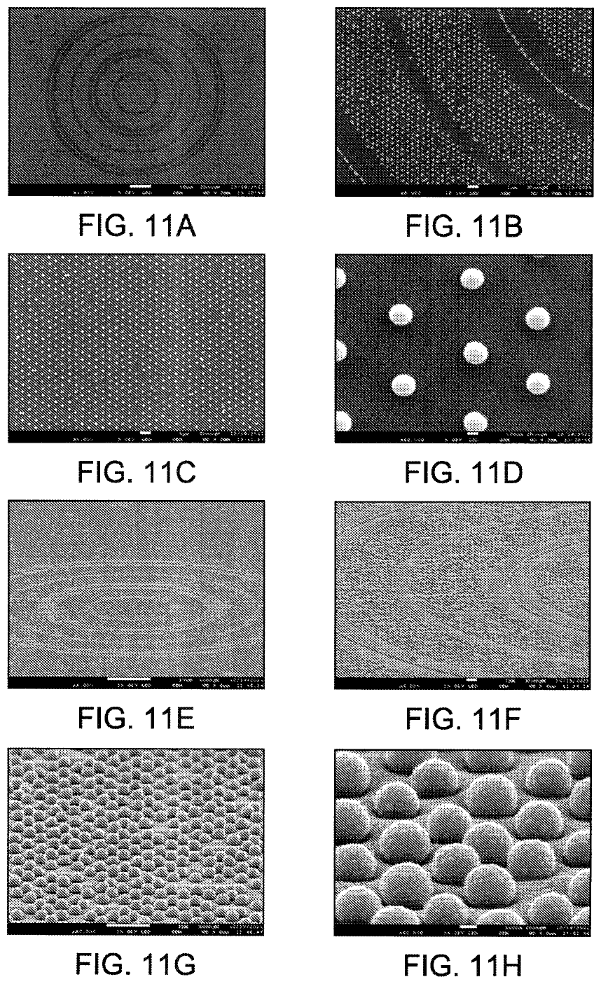

The flow cell channels 26 were washed with water flow to remove any non-specific bonded particles, leaving one pre-clustered particle 10′ captured by one chemical pad 22. Scanning electron micrographs of different portions of the flow cell surface after the pre-clustered particles 10′ were captured are shown in FIG. 11A through FIG. 11H. These images clearly illustrate successful capture of the pre-clustered particles 10′ at most of the flow cell chemical pads.

### Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such values or sub-ranges were explicitly recited. For example, a range from about 2 mm to about 300 mm, should be interpreted to include not only the explicitly recited limits of from about 2 mm to about 300 mm, but also to include individual values, such as about 40 mm, about 250.5 mm, etc., and sub-ranges, such as from about 25 mm to about 175 mm, etc.

Furthermore, when “about” and/or “substantially” are/is utilized to describe a value, they are meant to encompass minor variations (up to +/−10%) from the stated value.

While several examples have been described in detail, it is to be understood that the disclosed examples may be modified. Therefore, the foregoing description is to be considered non-limiting.

What is claimed is:

1. A sequencing kit, comprising:
a plurality of particles including:
a primer set attached to a surface of each of the plurality of particles; and
a flow cell surface attachment mechanism attached to the surface of each of the plurality of particles, the flow cell surface attachment mechanism being selected from the group consisting of a capture primer, an alkene, an alkyne, biotin, and a charged polymer; and
a flow cell including a plurality of chemical pads that are spatially separated from one another on a substantially flat substrate surface, each of the chemical pads including chemistry to attach to the surface attachment mechanism.

2. The sequencing kit as defined in claim 1, wherein:
each of the plurality of particles includes a core and a hydrogel attached to the core; and
the primer set is attached to the hydrogel.

3. The sequencing kit as defined in claim 2, wherein:
the flow cell surface attachment mechanism of each of the plurality of particles is the alkene or the alkyne;
the alkene or the alkyne is a functional group of the hydrogel; and
each of the plurality of chemical pads is poly(N-(5-azidoacetamidylpentyl)acrylamide-co-acrylamide).

4. The sequencing kit as defined in claim 1, wherein:
the flow cell surface attachment mechanism of each of the plurality of particles is the capture primer;
the capture primer is one of the primers of the primer set;
each of the plurality of chemical pads is poly(N-(5-azidoacetamidylpentyl)acrylamide-co-acrylamide); and
the sequencing kit further comprises an azide reducing agent to convert at least some azide groups of the poly(N-(5-azidoacetamidylpentyl)acrylamide-co -acrylamide) to amine groups that can attach to a 3′ end of the capture primer.

5. The sequencing kit as defined in claim 1, wherein:
the flow cell surface attachment mechanism of each of the plurality of particles is the biotin; and
each of the plurality of chemical pads is streptavidin.

6. The sequencing kit as defined in claim 1, wherein:
the flow cell surface attachment mechanism of each of the plurality of particles is the charged polymer; and
each of the plurality of chemical pads includes a counter ion of the charged polymer.

7. The sequencing kit as defined in claim 6, wherein:
the charged polymer is selected from the group consisting of polylysine, polyethylenimine and polypeptide; and
the counter ion is selected from the group consisting of an oligonucleotide, polyacrylic acid, and polystyrene sulfonate.

8. The sequencing kit as defined in claim 1, wherein:
the flow cell surface attachment mechanism of each of the plurality of particles is the capture primer;
the capture primer is CCTCCTCCTCCTCCTCCTCCTCCT (SEQ. ID. NO. 15;
each of the plurality of chemical pads includes a complementary primer of the capture primer.

9. A method, comprising:
amplifying a plurality of library fragments on respective surfaces of a plurality of particles, thereby generating pre-clustered particles, each of the plurality of particles including a surface attachment mechanism selected from the group consisting of a primer, an alkene, an alkyne, biotin, and a charged polymer; and
introducing the pre-clustered particles to a flow cell including a plurality of chemical pads that are spatially separated from one another on a substantially flat substrate surface, each of the chemical pads including chemistry to attach to the surface attachment mechanism.

10. A method, comprising:
generating a plurality of chemical pads that are spatially separated from one another on a substantially flat surface of a substrate, wherein each of the chemical pads includes poly(N-(5-azidoacetamidylpentyl)acrylamide-co-acrylamide); and
exposing the plurality of chemical pads to an azide reducing agent to convert at least some azide groups of the poly(N-(5-azidoacetamidylpentyl)acrylamide-co -acrylamide) to amine groups.

11. The method as defined in claim 10, wherein the azide reducing agent is selected from the group consisting of a phosphine and a phosphite.

12. The method as defined in claim 11, wherein:
the azide reducing agent is the phosphine selected from the group consisting of Tris(2-carboxyethyl)phosphine hydrochloride) (TCEP) and Tris(hydroxypropyl)phosphine; and
exposing the plurality of chemical pads to the azide reducing agent takes place at a temperature ranging from about 50° C. to about 60° C. for a time ranging from about 5 minutes to about 10 minutes.

13. The method as defined in claim 10, wherein generating the plurality of chemical pads involves:
applying a sacrificial layer over the substantially flat surface;
applying a resin layer over the sacrificial layer;
patterning the resin layer to include concave regions separated by convex regions;
removing the resin layer and the sacrificial layer from the concave regions, thereby exposing the substantially flat surface at the concave regions;
applying the poly(N-(5-azidoacetamidylpentyl)acrylamide-co-acrylamide) over the substantially flat surface at the concave regions and over the convex regions; and
lifting off remaining portions of the sacrificial layer, thereby removing the resin layer and the poly(N-(5-azidoacetamidylpentyl)acrylamide-co-acrylamide) that overlie the sacrificial layer.

14. The method as defined in claim 10, wherein generating the plurality of chemical pads involves:
applying the poly(N-(5-azidoacetamidylpentyl)acrylamide-co-acrylamide) over the substantially flat surface;
applying a sacrificial layer over the poly(N-(5-azidoacetamidylpentyl)acrylamide-co-acrylamide);
applying a resin layer over the sacrificial layer;
patterning the resin layer to include concave regions separated by convex regions;
removing the resin layer, the sacrificial layer, and the poly(N-(5-azidoacetamidylpentyl)acrylamide-co-acrylamide) from the concave regions, thereby exposing the substantially flat surface at the concave regions; and
lifting off remaining portions of the sacrificial layer, thereby removing the resin layer that overlies the sacrificial layer.

15. The method as defined in claim 14, wherein removing the resin layer, the sacrificial layer, and the poly(N-(5-azidoacetamidylpentyl)acrylamide-co -acrylamide) from the concave regions involves:
anisotropically etching the resin layer, the sacrificial layer, and the poly(N-(5-azidoacetamidylpentyl)acrylamide-co-acrylamide) from the concave regions; and
generating an undercut profile by isotropically etching some of the sacrificial layer and the poly(N-(5-azidoacetamidylpentyl)acrylamide-co-acrylamide) that underlie the resin layer at the convex regions.

16. The method as defined in claim 10, wherein generating the plurality of chemical pads involves:
applying the poly(N-(5-azidoacetamidylpentyl)acrylamide-co-acrylamide) over the substantially flat surface;
applying a sacrificial layer over the poly(N-(5-azidoacetamidylpentyl)acrylamide-co-acrylamide);
patterning the sacrificial layer to include concave regions separated by convex regions;
removing the sacrificial layer and the poly(N-(5-azidoacetamidylpentyl)acrylamide-co-acrylamide) from the concave regions, thereby exposing the substantially flat surface at the concave regions; and
lifting off remaining portions of the sacrificial layer.

17. The method as defined in claim 10, wherein generating the plurality of chemical pads involves:
using photolithography to generate a plurality of sacrificial pads on the substantially flat surface such that regions of the substantially flat surface separate each of the plurality of sacrificial pads;
applying the poly(N-(5-azidoacetamidylpentyl)acrylamide-co-acrylamide) on the plurality of spatially separated sacrificial pads and on the regions of the substantially flat surface;
introducing ultraviolet light through the substrate, whereby portions of the poly(N-(5-azidoacetamidylpentyl)acrylamide-co-acrylamide) overlying the regions of the substantially flat surface are cured and other portions of the poly(N-(5-azidoacetamidylpentyl)acrylamide-co-acrylamide) overlying the plurality of spatially separated sacrificial pads are uncured;
removing the uncured portions of the poly(N-(5-azidoacetamidylpentyl)acrylamide-co-acrylamide); and
removing the plurality of sacrificial pads.
